SEQUENCE LISTING (24 sequences)

SEQ ID NO: 1 - DNA, 20 bp, Mus musculus
gtttgccaagctgctgaagc

SEQ ID NO: 2 - DNA, 20 bp, Mus musculus
gaagcgacagatggtggtct

SEQ ID NO: 3 - DNA, 24 bp, Mus musculus
ctcaccctgggcgttctctttgga

SEQ ID NO: 4 - DNA, 22 bp, Mus musculus
caaggcctacgaacaggtcatg

SEQ ID NO: 5 - DNA, 26 bp, Mus musculus
ctccttggtagtaggaagtgtctgca

SEQ ID NO: 6 - DNA, 20 bp, Mus musculus
agcctggatgcctcgcccct

SEQ ID NO: 7 - DNA, 20 bp, Mus musculus
gtaggcctctcctgtgccaa

SEQ ID NO: 8 - DNA, 20 bp, Mus musculus
tacatacaggctcaccctcg

SEQ ID NO: 9 - DNA, 24 bp, Mus musculus
tgccagactaccaccaccacgctg

SEQ ID NO: 10 - DNA, 17 bp, Mus musculus
ggccgatgaacgccttt

SEQ ID NO: 11 - DNA, 19 bp, Mus musculus
tctgggttctgctgtgcca

SEQ ID NO: 12 - DNA, 23 bp, Mus musculus
cttgcgttcgtctttggcccaca

SEQ ID NO: 13 - DNA, 20 bp, Mus musculus
attgcctccacgtgctgcca

SEQ ID NO: 14 - DNA, 20 bp, Mus musculus
gaaaatgtcggccattccct

SEQ ID NO: 15 - DNA, 23 bp, Mus musculus
cagccggaccatttctcctcgct

SEQ ID NO: 16 - DNA, 20 bp, Mus musculus
ggcaccaagtgttgtacact

SEQ ID NO: 17 - DNA, 20 bp, Mus musculus
agcagacacacacggttcag

SEQ ID NO: 18 - DNA, 26 bp, Mus musculus
ccttgtgtggaggactatctgtctgc

SEQ ID NO: 19 - DNA, 20 bp, Mus musculus
atcctggtgtaccgcctgaa

SEQ ID NO: 20 - DNA, 20 bp, Mus musculus
tgctggatgaagtggtactc

SEQ ID NO: 21 - DNA, 21 bp, Mus musculus
cagctgccacatgcgcaccct

SEQ ID NO: 22 - DNA, 17 bp, Mus musculus
gaagcgagcctttggta

SEQ ID NO: 23 - DNA, 23 bp, Mus musculus
tgaacttgagttatggtacctca

SEQ ID NO: 24 - DNA, 24 bp, Mus musculus
ctccgctcatcttcccagttcacc

(19) United States
(12) Patent Application Publication
(10) Pub. No.: US 2009/0010900 A1
(43) Pub. Date: Jan. 8, 2009

(54) Embryonic Stem Cell Derivatives, and Methods of Making and Using the Same

(75) Inventors: Jeffrey H. Fair, Chapel Hill, NC (US); Bruce Cairns, Pittsboro, NC (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: THE UNIVERSITY OF NORTH CARLOLINA AT CHAPEL HILL, Chapel Hill, NC (US)

(21) Appl. No.: 11/596,247

(22) PCT Filed: Jun. 30, 2005
(86) PCT No.: PCT/US2005/023455

§ 371 Date: Jul. 31, 2008

Related U.S. Application Data
(60) Provisional application No. 60/586,000, filed on Jul. 7, 2004.

(51) Int. Cl. A61K 35/12; C12N 5/06; A61P 1/00

(52) U.S. Cl. 424/93.21; 424/93.7; 435/377; 435/366

(57) ABSTRACT

The presently disclosed subject matter provides embryonic stem (ES) cell derivatives that can be employed in therapeutic methods. Also provided are methods for generating and using the ES cell derivatives.

### CROSS REFERENCE TO RELATED APPLICATIONS

This application is based on and claims priority to U.S. Provisional Patent Application Ser. No. 60/586,000, filed Jul. 7, 2004, the entire disclosure of which is herein incorporated by reference.

### GRANT STATEMENT

This work was supported by grant numbers K18 DK065013 and K08 GM067147 from the U.S. National Institutes of Health. Thus, the U.S. government has certain rights in the presently disclosed subject matter.

### TECHNICAL FIELD

The presently disclosed subject matter generally relates to embryonic stem (ES) cell derivatives that can be employed in therapeutic methods. Also provided are methods for making and using the ES cell derivatives.

TABLE OF ABBREVIATIONS

| | |
|---|---|
| aFGF | acidic fibroblast growth factor |
| Afp | α-fetoprotein |
| Alb | albumin |
| BSA | bovine serum albumin |
| DMEM | Dulbecco's modified Eagle's medium |
| EDTA | ethylene diamine tetraacetic acid |
| ES | embryonic stem |
| F-IX | Factor IX |
| FGF | fibroblast growth factor |
| GFP | green fluorescent protein |
| Hprt | hypoxanthine phosphoribosyl transferase |
| HrGn | humanized renilla green (a GFP) |
| i.m. | intramuscular(ly) |
| LCM | Laser-Capture Microdissection |
| LIF | Leukemia Inhibitory Factor |
| MAT1A | Methionine Adenosyltransferase 1A |
| PBS | phosphate-buffered saline |
| PEP(s) | putative endodermal precursor(s) |
| s.c. | subcutaneous(ly) |
| Tty | transthyretin |

### BACKGROUND

Embryonic stem (ES) cells, derived from the inner cell mass of the mammalian blastocyst, can be induced to undergo differentiation into all cell types of the organism, thus leading to intense interest in their potential use for therapeutic applications. Despite this interest, significant problems have arisen in translating the potential of ES-derived cells into clinical reagents that can restore tissue function or reverse human disease.

Part of the difficulty is that the ES cell is an artificial construct requiring maintenance of complex transcriptional regulation not normally found in vivo. In addition, regulation of differentiation in vitro is complex and differs from differentiation that normally occurs during embryogenesis and subsequent development. Additionally, once in vitro differentiation has occurred, transplantation of the ES-derived cells in subjects can lead to additional problems, including teratoma formation and alloimmunity. Yet the potential for ES-derived cells to significantly alleviate the burden of disease is substantial as ES-derived cells successfully engrafted within a target tissue could serve as “gene replacement vectors” that conceivably could reverse acute or chronic disorders and/or repair a myriad of metabolic defects.

While hepatocyte phenotypes can be derived from ES cells in vitro, most strategies of hepatic lineage ES cell differentiation have relied on isolation of cells from post-embryoid body cultures. Unfortunately, achieving robust engraftment with, and subsequent function of, cells generated in this manner has proven to be difficult.

What are needed, then, are methods for producing ES cell-derived in vitro differentiated cells that can be transplanted into subjects to provide treatment for various diseases, and methods of employing such ES cell-derived in vitro differentiated cells for treating disease. The presently disclosed subject matter addresses these and other needs in the art in whole or in part.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter provides methods of treating a disease associated with abnormal or missing function of a tissue in a subject. In some embodiments, the method comprises (a) differentiating an embryonic stem cell or a pre-embryoid body cell comprising the gene of interest in vitro to form a putative endoderm cell (PEP); and (b) introducing into the subject the PEP, whereby the PEP becomes stably engrafted in an endoderm-derived tissue of the subject and provides the abnormal or missing function to the subject. In some embodiments, the endoderm-derived tissue of the subject is liver. In some embodiments, the introducing is into liver parenchyma of the subject. In some embodiments, a disease associated with abnormal or missing function of a tissue in a subject results from abnormal or absent function of a cell type of the tissue or abnormal or absent expression of a gene of interest in the tissue.

In some embodiments, the presently disclosed compositions and methods can be used to deliver any nucleic acid (e.g., any gene of interest or regulatory sequence) to a target tissue. In some embodiments, the gene of interest is normally expressed in liver cells in the absence of the disease. In some embodiments, the gene of interest is a Factor IX gene.

In some embodiments of the disclosed methods, the differentiating step comprises exposing the embryonic stem cell or the pre-embryoid body cell to a growth factor. In some embodiments, the growth factor is acidic fibroblast growth factor (aFGF). In some embodiments, the differentiating step results in reduced expression of Oct4 and increased expression of an endodermal marker selected from the group consisting of FoxA2 (Hnf-β3), Gata4, Sox17α, albumin (Alb), and α-fetoprotein (Afp) in the in vitro differentiated cell.

In some embodiments, the in vitro differentiated cell is syngeneic to the subject and in some embodiments the in vitro differentiated cell is allogeneic to the subject. In some embodiments, the in vitro differentiated cell further comprises a coding sequence that is not normally expressed in the liver of the subject in the absence of disease, but instead is normally expressed in another tissue or cell type in the subject in the absence of disease.

The presently disclosed subject matter also provides a method for producing a reagent comprising an in vitro differentiated cell. In some embodiments, the method comprises (a) providing an embryonic stem (ES) cell or a pre-embryoid body cell; and (b) differentiating the ES cell or the pre-embryoid body cell in vitro to express a marker associated with a differentiated cell of interest, wherein the differentiated cell of interest normally expresses a gene of interest. In some embodiments, the embryonic stem cell or the pre-embryoid body cell is a mammalian cell. In some embodiments, the mammalian cell is a human cell.

In some embodiments, the differentiating comprises exposing the embryonic stem cell or the pre-embryoid body cell to a growth factor in an amount and for a time sufficient to cause the embryonic stem cell to reduce expression of Oct4 and increase expression of an endodermal marker selected from the group consisting of FoxA2 (Hnf-β3), Gata4, Sox17α, albumin, and α-fetoprotein. In some embodiments, the growth factor is an acidic fibroblast growth factor (aFGF).

The presently disclosed subject matter also provides a method for generating a putative endoderm cell (PEP) from an embryonic stem cell. In some embodiments, the method comprises (a) culturing the embryonic stem cell in the absence of feeder cells and LIF; and (b) exposing the embryonic stem cell to acidic fibroblast growth factor in an amount and for a time sufficient to cause the embryonic stem cell to reduce expression of Oct4 and to increase expression of an mRNA selected from the group consisting of α-fetoprotein, Gata4, albumin, FoxA2 (HNF-β3), and Sox 17α, whereby a putative endoderm cell is generated. In some embodiments, the embryonic stem cell is a mammalian embryonic stem cell. In some embodiments, the embryonic stem cell is a human embryonic stem cell. In some embodiments, the embryonic stem cells are exposed to acidic fibroblast growth factor in an amount of about 100 ng/ml in a culture medium for about 7 days. In some embodiments, the method further comprises purifying the putative endoderm cell.

The presently disclosed subject matter also provides a putative endoderm cell (PEP) produced by the disclosed methods.

The presently disclosed compositions and methods can be used to treat diseases in any subject. In some embodiments, the subject is a mammal. In some embodiments, the mammal is a human.

Thus, it is an object of the presently disclosed subject matter to provide compositions and methods that can be used to treat diseases in a subject.

An object of the presently disclosed subject matter having been stated above, other objects and advantages of the presently disclosed subject matter will become apparent to those of ordinary skill in the art after a study of the following description and non-limiting Examples.

### BRIEF DESCRIPTION OF THE DRAWINGS

FIGS. 1A and 1B depict analyses of in vitro differentiated ES-derived putative endodermal precursors (PEPs). FIG. 1A depicts the results of real-time quantitative PCR analysis of gene expression of Oct4, FoxA2, Sox17, Gata4, α-fetoprotein (Afp), and albumin (Alb) of PEPs at 0, 4, and 7 days after in vitro differentiation in 100 ng/ml acidic fibroblast growth factor (aFGF) in media. FIG. 1B depicts the appearances of undifferentiated and differentiated ES cells. The top panel depicts the appearance of undifferentiated ES cells growing on a feeder layer. The bottom two panels depict the appearances of ES cells after spontaneous differentiation (bottom left) or after differentiation with aFGF (bottom right) for 7 days.

FIGS. 2A-2D depict the results of in vivo engraftment studies using the in vitro differentiated ES-derived cells. FIG. 2A depicts a typical engraftment at 20 days as detected by 3-D stereomicroscopy on a fresh liver section. FIGS. 2B and 2C depict engraftment images of the same specimen as depicted in FIG. 2A after histological sectioning. FIG. 2B depicts an image as seen through a green fluorescent protein (GFP) filter only, and FIG. 2C depicts the same image as seen through a GFP filter with a light microscopy overlay showing hepatic architecture. FIG. 2D depicts before (left panel) and after (right panel) images of laser capture microdissection of engraftment in a frozen section. The burn in the center of the “before” image represents the beginning of laser capture.

FIG. 3 depicts plasma Factor IX (F-IX) concentration levels over time in F-IX knockout mice engrafted with in vitro differentiated ES-derived cells. The results of six different engraftment experiments are shown using two different genetic backgrounds. Mice Nos. 1-3 received partial hepatectomy before injection of PEPs, and three mice (mice numbered 4-6 in FIG. 3) received PEP injection without hepatectomy. Mouse No. 2 died during anesthesia for blood drawing at day 10.

FIGS. 4A-4D depict confocal analyses of immunofluorescence staining of liver sections from BALB/c F-IX knockout mice at day 115 after injection. For each of FIGS. 4A-4D, the four panels (left to right) are each of the same section, and depict the following: (i) the first panel depicts the results of staining a liver section with a sheep anti-rat F-IX antibody followed by detection with a Texas red-labeled anti-sheep immunoglobulin (Ig) (Fab′2) IgG secondary antibody; (ii) the second panel depicts the results of staining the same liver section with a rabbit polyclonal anti-GFP antibody conjugated to Alexa Fluor 488; (iii) the third panel depicts phase contrast microscopy of the liver section; and (iv) the fourth panel depicts merged phase overlay images of the first three panels.

FIG. 4A depicts liver sections from wild type mice without PEP injection, demonstrating F-IX staining and no GFP staining. FIGS. 4B and 4C depict liver sections from two different F-IX knockout mice 115 days after PEP injection, each demonstrating F-IX staining and GFP staining, with co-localization of F-IX and GFP staining demonstrated on the phase overlay images. FIG. 4D depicts liver sections from F-IX knockout mice without PEP injection, demonstrating neither F-IX staining nor GFP staining (magnification: x63).

FIGS. 5A-5C depict genetic analyses of laser capture microdissected PEP hepatic engraftment. FIGS. 5A and 5B depict a schematic representation of the wild type Hprt locus (FIG. 5A), and an Hprt locus that has been interrupted with HrGn (humanized renilla green; GFP) sequences by homologous recombination (FIG. 5B). Wild type hypoxanthine phosphoribosyl transferase (Hprt) sequences are shown by black bars and donor GFP-modified Hprt locus sequences are shown by white bars. FIG. 5C depicts the results of genetic analysis of laser capture microdissected PEP hepatic engraftment from three engraftment specimens (specimens 1, 2, and 3) with wild type liver and GFP liver serving as controls. For the data presented in the bar graph, the wild type sample was set at 100 relative DNA copy numbers for wild type DNA and GFP positive sample was assumed to be 100 relative DNA copy numbers for GFP DNA.

### BRIEF DESCRIPTION OF THE SEQUENCE LISTING

SEQ ID NOs: 1-24 are the sequences of oligonucleotides that were used to perform quantitative RT-PCR of PEPs and isolated liver cells to determine the expression of Oct4, FoxA2, Gata4, Sox17α, Afp, and Alb (SEQ ID NOs: 1-18) or to genotype cells based on the relative content of GFP-(SEQ ID NO: 19-21) and Hprt-(SEQ ID NOs: 22-24) encoding DNA.

More specifically, SEQ ID NOs: 1-3 are the nucleotide sequences of oligonucleotides specific for the murine Oct4 gene, with SEQ ID NOs: 1 and 2 being the forward and reverse primers, respectively, for the quantitative RT-PCR reaction, and SEQ ID NO: 3 being the sequence of an Oct4-specific fluoroprobe.

SEQ ID NOs: 4-6 are specific for the murine FoxA2 gene, with SEQ ID NOs: 4 and 5 being the forward and reverse primers, respectively, for the quantitative RT-PCR reaction, and SEQ ID NO: 6 being the sequence of an FoxA2-specific fluoroprobe.

SEQ ID NOs: 7-9 are the nucleotide sequences of oligonucleotides specific for the murine Gata4 gene, with SEQ ID NOs: 7 and 8 being the forward and reverse primers, respectively, for the quantitative RT-PCR reaction, and SEQ ID NO: 9 being the sequence of an Gata4-specific fluoroprobe.

SEQ ID NOs: 10-12 are the nucleotide sequences of oligonucleotides specific for the murine Sox17α gene, with SEQ ID NOs: 10 and 11 being the forward and reverse primers, respectively, for the quantitative RT-PCR reaction, and SEQ ID NO: 12 being the sequence of an Sox17α-specific fluoroprobe.

SEQ ID NOs: 13-15 are the nucleotide sequences of oligonucleotides specific for the murine Afp gene, with SEQ ID NOs: 13 and 14 being the forward and reverse primers, respectively, for the quantitative RT-PCR reaction, and SEQ ID NO: 15 being the sequence of an Afp-specific fluoroprobe.

SEQ ID NOs: 16-18 are the nucleotide sequences of oligonucleotides specific for the murine Alb gene, with SEQ ID NOs: 16 and 17 being the forward and reverse primers, respectively, for the quantitative RT-PCR reaction, and SEQ ID NO: 18 being the sequence of an Alb-specific fluoroprobe.

SEQ ID NOs: 19-21 are the nucleotide sequences of oligonucleotides specific for the green fluorescent protein (HrGn) gene, with SEQ ID NOs: 19 and 20 being the forward and reverse primers, respectively, for the quantitative PCR reaction, and SEQ ID NO: 21 being the sequence of an HrGn-specific fluoroprobe.

SEQ ID NOs: 22-24 are the nucleotide sequences of oligonucleotides specific for the murine Hprt gene, with SEQ ID NOs: 22 and 23 being the forward and reverse primers, respectively, for the PCR reaction, and SEQ ID NO: 24 being the sequence of an Hprt-specific fluoroprobe.

### DETAILED DESCRIPTION

The presently disclosed subject matter will be now be described more fully hereinafter with reference to the accompanying Examples, in which representative embodiments of the presently disclosed subject matter are shown. The presently disclosed subject matter can, however, be embodied in different forms and should not be construed to be limited to the embodiments set forth herein. Rather, these embodiments are provided so that this disclosure will be thorough and complete, and will fully convey the scope of the presently disclosed subject matter to those skilled in the art.

All publications, patent applications, patents, and other references mentioned herein are incorporated by reference in their entireties.

### I. General Considerations

Embryonic stem (ES) cells are undifferentiated, pluripotent cells derived in vitro from preimplantation embryos (Evans et al., 1981; Martin, 1981). Mammalian ES cells maintain an undifferentiated state through serial passages when cultured in the presence of fibroblast feeder layers and in the presence of Leukemia Inhibitory Factor (LIF; see Williams et al., 1988; Smith et al., 1988; Gearing et al., 1989; Pease et al., 1990). If LIF is removed, ES cells spontaneously differentiate.

Mammalian ES cells cultured under non-attaching conditions aggregate and differentiate into simple embryoid bodies, with an outer layer of endoderm and an inner core of primitive ectoderm. If these embryoid bodies are then allowed to attach onto a tissue culture surface, disorganized differentiation of various cell types occurs (Martin, 1981; Doetschman et al., 1985).

ES cells differentiate in vitro into all cell types of the organism, and ES-derived cells selected for hepatic lineages have been shown in various models to engraft into liver in vivo. However, previous studies of ES cell liver engraftment have only demonstrated limited, short-term function without persistent mature hepatocyte function that restores a liver specific synthetic defect. The presently disclosed subject matter provides for the generation of ES-derived cells in vitro by direct pre-embryoid body differentiation, and the resulting cells, referred to herein as “putative endodermal precursors” (PEPs), display an endodermal gene expression profile characterized in some embodiments as a profile associated with low expression of the pluripotency transcription factor Oct4.

When injected into a murine model of two-thirds partial hepatectomy, PEPs robustly engrafted into the sinusoidal architecture and displayed persistent hepatocyte-specific synthetic function with expression of intact murine Factor IX (F-IX) protein in a F-IX knockout mouse. Genetic analysis of specimens isolated by laser capture microdissection indicated that engraftment was not the result of cell fusion, and long term engraftment and function was maintained across a major MHC mismatch barrier without immunosuppressive conditioning of the recipient.

Thus, the presently disclosed subject matter relates to in vitro differentiated ES cells that give rise to PEPs, which thereafter can be used to treat various medical conditions, including but not limited to those medical conditions associated with abnormal or absent gene expression. While the experiments disclosed herein relate to a rescue of a clotting defect in mice that lack Factor IX, the methods and compositions disclosed herein are not limited to treating Factor IX deficiency, as PEPs would be expected to be able to express any gene, including but not limited to genes that are normally present in the genome of the ES cells as well as transgenes that have been incorporated into the genomes of the ES cells.

### II. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims. Thus, for example, reference to “a cell” (e.g., “a PEP”) includes a plurality of such cells (e.g., a plurality of PEPs), and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

As used herein, the term “cell” refers not only to the particular subject cell (e.g., a living biological cell such as an ES cell or a PEP), but also to the progeny or potential progeny of such a cell. Because certain modifications can occur in succeeding generations due to either mutation or environmental influences, such progeny might not, in fact, be identical to the parent cell, but are still included within the scope of the term as used herein.

As used herein, the phrase “differentiating in vitro” and grammatical variants thereof refers to an in vitro modification of a cell (e.g., an ES cell or a pre-embryoid body cell) that results in the cell expressing markers consistent with different cell types. For example, ES cells are undifferentiated, pluripotent cells derived in vitro from preimplantation embryos. ES cells maintain an undifferentiated state through serial passages when cultured in the presence of fibroblast feeder layers and/or in the presence of Leukemia Inhibitory Factor (LIF; purified LIF human and murine are available from CHEMICON® International, Inc., Temecula, Calif., United States of America).

Thus, in vitro differentiation of an ES cell can result in the ES cell becoming restricted in its ability to develop into various cell types (i.e., losing its pluripotency). In some embodiments of the presently disclosed subject matter, ES cells or cells derived from pre-embryoid body culture are differentiated in vitro to express certain markers of cells of endodermal origin by growing the cells in the presence of an inducing agent such as, but not limited to chicken cardiac mesoderm cells or acidic fibroblast growth factor.

Mouse ES cells cultured under non-attaching conditions aggregate and differentiate into simple embryoid bodies, with an outer layer of endoderm and an inner core of primitive ectoderm. If these embryoid bodies are then allowed to attach onto a tissue culture surface, disorganized differentiation of various cell types, including nerves, blood cells, muscle, and cartilage, occurs.

Thus, as used herein the term “pre-embryoid body cell” refers to an ES cell or a derivative of an ES cell that has been cultured under conditions sufficient to substantially prevent the formation of embryoid bodies and further that has not formed a part of an embryoid body. Typically, conditions sufficient to substantially prevent the formation of embryoid bodies include maintaining the ES cells on an appropriate feeder layer and/or culturing the ES cells in the presence of certain growth factors such as LIF, to maintain ES cells in an undifferentiated state until steps toward differentiation are desired. It is understood, however, that a method used to induce in vitro differentiation can include steps that are normally associated with embryoid body formation (for example, removal from feeders and/or growth of the cells in the absence of LIF) provided that the cells are not permitted to form embryoid bodies.

The term “gene” refers broadly to any segment of DNA associated with a biological function. A gene typically encompasses one or more sequences selected from the group consisting of a coding sequence, a promoter region, a transcriptional regulatory sequence, a non-expressed DNA segment that is a specific recognition sequence for regulatory proteins, a non-expressed DNA segment that contributes to gene expression, a DNA segment designed to have desired parameters, and combinations thereof. A gene can be obtained by a variety of methods, including isolation or cloning from a biological sample, synthesis based on known or predicted sequence information, and recombinant derivation of an existing sequence. As such, the term “gene” refers to a transcription unit.

In some embodiments, however, the term “gene” is used interchangeably with “coding sequence” and/or “open reading frame” to refer to a protein coding subsequence of a genetic locus. Thus, the term “gene” can be used to refer to an entire genetic locus including a coding sequence, introns, and operatively linked regulatory elements, or it can be used to refer to the coding sequences of a genetic locus without operatively linked regulatory elements (e.g., the exons without or without introns, if present), or it can refer to a spliced or unspliced transcription product of the genetic locus (for example, an mRNA), depending on the context in which the term is employed. In some embodiments, the term “gene” refers to a cDNA.

As used herein, the term “interact” includes “binding” interactions and “associations” between molecules. Interactions can be, for example, protein-protein, protein-small molecule, protein-nucleic acid, and nucleic acid-nucleic acid in nature.

As used herein, the term “modulate” refers to an increase, decrease, or other alteration of any, or all, chemical and biological activities or properties of a biochemical entity, e.g., a wild type or mutant polypeptide. As such, the term “modulate” can refer to a change in the expression level of a gene (or a level of RNA molecule or equivalent RNA molecules encoding one or more proteins or protein subunits), or of an activity of one or more proteins or protein subunits, such that expression, level, or activity is greater than or less than that observed in the absence of the modulator. For example, the term “modulate” can mean “inhibit” or “suppress”, but the use of the word “modulate” is not limited to this definition.

Thus, the term “modulation” as used herein refers to both upregulation (i.e., activation or stimulation) and downregulation (i.e., inhibition or suppression) of a response. When used in reference to a functional property or biological activity or process (e.g., enzyme activity or receptor binding), the term “modulate” refers to the capacity to upregulate (e.g., activate or stimulate), downregulate (e.g., inhibit or suppress), or otherwise change a quality of such property, activity, or process. In certain instances, such regulation can be contingent on the occurrence of a specific event, such as activation of a signal transduction pathway, and/or can be manifest only in particular cell types.

The term “modulator” refers to a polypeptide, nucleic acid, macromolecule, complex, molecule, small molecule, compound, species, or the like, naturally or non-naturally occurring, which can be capable of causing modulation under a given set of conditions. Modulators can be evaluated for potential activity as inhibitors or activators (directly or indirectly) of a functional property, biological activity or process, or a combination thereof, (e.g., agonist, partial antagonist, partial agonist, inverse agonist, antagonist, and the like) by inclusion in assays. In such assays, many modulators can be screened at one time. The activity of a modulator can be known, unknown, or partially known. In some embodiments, a modulator refers to a chemical substance that inactivates or decreases a biological activity of a biomolecule such as a biosynthetic and catalytic activity, a receptor, a signal transduction polypeptide, a structural gene product, or a transport polypeptide.

Modulators can be either selective or non-selective. As used herein, the term “selective” when used in the context of a modulator (e.g., an inhibitor) refers to a measurable or otherwise biologically relevant difference in the way the modulator interacts with one molecule (e.g., an enzyme or receptor) versus another similar but not identical molecule (e.g., a member of the same enzyme or receptor family).

It must be understood that it is not required that the degree to which the interactions differ be completely opposite. Put another way, the term selective modulator encompasses not only those molecules that only bind to a given polypeptide and not to related family members. The term is also intended to include modulators that are characterized by interactions with polypeptides of interest and from related family members that differ to a lesser degree. For example, selective modulators include modulators for which conditions can be found (such as the nature of the substituents present on the modulator) that would allow a biologically relevant difference in the binding of the modulator to the polypeptide of interest versus polypeptides derived from different family members.

When a selective modulator is identified, the modulator will bind to one molecule (for example a polypeptide of interest) in a manner that is different (for example, stronger) than it binds to another molecule (for example, a polypeptide related to the polypeptide of interest). As used herein, the modulator is said to display “selective binding” or “preferential binding” to the molecule to which it binds more strongly.

As used herein, the term “mutation” carries its traditional connotation and means a change, inherited, naturally occurring or introduced, in a nucleic acid or polypeptide sequence, and is used in its sense as generally known to those of skill in the art.

As used herein, the terms “nucleic acid” and “nucleic acid molecule” mean any of deoxyribonucleic acid (DNA), ribonucleic acid (RNA), oligonucleotides, fragments generated by the polymerase chain reaction (PCR), and fragments generated by any of ligation, scission, endonuclease action, and exonuclease action. Nucleic acids can be composed of monomers that are naturally occurring nucleotides (such as deoxyribonucleotides and ribonucleotides), or analogs of naturally occurring nucleotides (e.g., α-enantiomeric forms of naturally-occurring nucleotides), or a combination of both. Nucleic acids can be either single stranded or double stranded.

The terms “operatively linked” and “operably linked”, as used herein, refer to a promoter region that is connected to a nucleotide sequence (for example, a coding sequence or open reading frame) in such a way that the transcription of the nucleotide sequence is controlled and regulated by that promoter region. Similarly, a nucleotide sequence is said to be under the “transcriptional control” of a promoter to which it is operably linked. Techniques for operatively linking a promoter region to a nucleotide sequence are known in the art.

The terms “heterologous gene”, “heterologous DNA sequence”, “heterologous nucleotide sequence”, “exogenous nucleic acid molecule”, and “exogenous DNA segment”, as used herein, refer to a sequence that originates from a source foreign to an intended host cell or, if from the same source, is modified from its original form. Thus, a heterologous gene in a host cell includes a gene that is endogenous to the particular host cell but has been modified, for example by mutagenesis or by isolation from native transcriptional regulatory sequences. The terms also include non-naturally occurring multiple copies of a naturally occurring nucleotide sequence.

Thus, the terms refer to a DNA segment that is foreign or heterologous to the cell, or homologous to the cell but in a position within the host cell nucleic acid wherein the element is not ordinarily found. In some embodiments where the heterologous DNA sequence comprises an open reading frame, the heterologous DNA sequence is also referred to as a “transgene”, although the term “transgene” is not limited to heterologous DNA sequences that comprise an open reading frame.

The term “expression vector” as used herein refers to a DNA sequence capable of directing expression of a particular nucleotide sequence in an appropriate host cell, comprising a promoter operatively linked to the nucleotide sequence of interest which is operatively linked to termination signals. It also typically comprises sequences required for proper translation of the nucleotide sequence. The construct comprising the nucleotide sequence of interest can be chimeric. The construct can also be one that is naturally occurring but has been obtained in a recombinant form useful for heterologous expression.

The term “promoter” or “promoter region” each refers to a nucleotide sequence within a gene that is positioned 5′ to a coding sequence of a same gene and functions to direct transcription of the coding sequence. The promoter region comprises a transcriptional start site, and can additionally include one or more transcriptional regulatory elements. In some embodiments, a method of the presently disclosed subject matter employs a promoter that is active in an endoderm-derived tissue. Exemplary such promoters include promoters that are active in the liver, the pancreas, the spleen, the lung, etc.

A “minimal promoter” is a nucleotide sequence that has the minimal elements required to enable basal level transcription to occur. As such, minimal promoters are not complete promoters but rather are subsequences of promoters that are capable of directing a basal level of transcription of a reporter construct in an experimental system. Minimal promoters include but are not limited to the CMV minimal promoter, the HSV-tk minimal promoter, the simian virus 40 (SV40) minimal promoter, the human β-actin minimal promoter, the human EF2 minimal promoter, the adenovirus E1B minimal promoter, and the heat shock protein (hsp) 70 minimal promoter. Minimal promoters are often augmented with one or more transcriptional regulatory elements to influence the transcription of an operably linked gene. For example, cell-type-specific or tissue-specific transcriptional regulatory elements can be added to minimal promoters to create recombinant promoters that direct transcription of an operably linked nucleotide sequence in a cell-type-specific or tissue-specific manner

Different promoters have different combinations of transcriptional regulatory elements. Whether or not a gene is expressed in a cell is dependent on a combination of the particular transcriptional regulatory elements that make up the gene's promoter and the different transcription factors that are present within the nucleus of the cell. As such, promoters are often classified as “constitutive”, “tissue-specific”, “cell-type-specific”, or “inducible”, depending on their functional activities in vivo or in vitro. For example, a constitutive promoter is one that is capable of directing transcription of a gene in a variety of cell types. Exemplary constitutive promoters include the promoters for the following genes which encode certain constitutive or “housekeeping” functions: hypoxanthine phosphoribosyl transferase (Hprt), dihydrofolate reductase (Dhfr; Scharfmann et al., 1991), adenosine deaminase, phosphoglycerate kinase (Pgk), pyruvate kinase, phosphoglycerate mutase, the β-actin promoter (see e.g., Williams et al., 1993), and other constitutive promoters known to those of skill in the art. “Tissue-specific” or “cell-type-specific” promoters, on the other hand, direct transcription in some tissues and cell types but are inactive in others. Exemplary tissue-specific promoters include the liver-specific promoters such as the transthyretin (Tty) promoter (Qian et al., 1995), ApoE (Simonet et al., 1993), and pancreas-specific promoters such as the promoter of the human pancreatic secretory trypsin inhibitor (PSTI; Yasuda et al., 1998), the SEL1L promoter (Cattaneo et al., 2001), the insulin promoter (Odagiri et al., 1996), as well as other tissue-specific and cell-type specific promoters known to those of skill in the art.

When used in the context of a promoter, the term “linked” as used herein refers to a physical proximity of promoter elements such that they function together to direct transcription of an operably linked nucleotide sequence

The term “transcriptional regulatory sequence” or “transcriptional regulatory element”, as used herein, each refers to a nucleotide sequence within the promoter region that enables responsiveness to a regulatory transcription factor. Responsiveness can encompass a decrease or an increase in transcriptional output and is mediated by binding of the transcription factor to the DNA molecule comprising the transcriptional regulatory element.

The term “transcription factor” generally refers to a protein that modulates gene expression by interaction with the transcriptional regulatory element and cellular components for transcription, including RNA Polymerase, Transcription Associated Factors (TAFs), chromatin-remodeling proteins, and any other relevant protein that impacts gene transcription.

The terms “reporter gene” and “marker gene” refer to an exogenous gene encoding a product that is readily observed and/or quantitated. A reporter gene is exogenous in that it originates from a source foreign to an intended host cell or, if from the same source, is modified from its original form. Non-limiting examples of detectable reporter genes that can be operatively linked to a transcriptional regulatory region can be found in Alam & Cook, 1990, and PCT International Publication No. WO 97/47763. Exemplary reporter genes include the lacZ gene (see e.g., Rose & Botstein, 1983), Green Fluorescent Protein (GFP; Cubitt et al., 1995), luciferase, and chloramphenicol acetyl transferase (CAT). Any suitable reporter and detection method can be used, and it will be appreciated by one of skill in the art that no particular choice is essential to or a limitation of the presently disclosed subject matter.

As used herein, the term “selectable marker” refers to a gene or gene product that confers a growth advantage to a cell that expresses it. For example, a selectable marker can allow a cell that expresses it to grow in the presence of a chemical (e.g., a drug such as G418) that would inhibit the growth of or kill cells that do not express the selectable marker. Selectable marker genes include, but are not limited to antibiotic resistance genes, for example the antibiotic resistance gene confers neomycin resistance (herein referred to as the “neo gene”).

As used herein, the term “polypeptide” means any polymer comprising any of the 20 protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless otherwise noted. As used herein, the terms “protein”, “polypeptide” and “peptide” are used interchangeably. The term “polypeptide” encompasses proteins of all functions, including enzymes.

As used herein, “significance” or “significant” relates to a statistical analysis of the probability that there is a non-random association between two or more occurrences. To determine whether or not a relationship is “significant” or has “significance”, statistical manipulations of the data can be performed to calculate a probability, expressed as a “p-value”. Those p-values that fall below a user-defined cutoff point are regarded as significant. In some embodiments, a p-value less than or equal to 0.10, in some embodiments less than or equal to 0.05, in some embodiments less than or equal to 0.01, in some embodiments less than or equal to 0.005, and in some embodiments less than or equal to 0.001, are regarded as significant.

As used herein, the term “significant increase”, and grammatical variants thereof, refers to an increase in activity (for example, a gene expression level) that is larger than the margin of error inherent in the measurement technique, in some embodiments an increase by about 2 fold or greater over a baseline activity (for example, the expression level of a gene in a PEP versus the expression level of the same gene in an ES cell, or vice versa), in some embodiments an increase by about 5 fold or greater, and in still some embodiments an increase by about 10 fold or greater. In some embodiments, a significant increase in gene expression refers to a level of gene expression that is below a detection limit in one cell (e.g., an ES cell) but is detectable in a derivative of that cell (e.g., a PEP).

As used herein, the terms “significantly less” and “significantly reduced”, and grammatical variants thereof, refer to a result (for example, a level of gene expression) that is reduced by more than the margin of error inherent in the measurement technique, in some embodiments a decrease by about 2 fold or greater with respect to a baseline activity (for example, the activity of the wild type enzyme in the absence of the inhibitor), in some embodiments, a decrease by about 5 fold or greater, and in still some embodiments a decrease by about 10 fold or greater. In some embodiments, a significant reduction in gene expression refers to a level of gene expression that is detectable in one cell (e.g., an ES cell) but is undetectable in a derivative of that cell (e.g., a PEP).

As used herein, the term “substantially”, when referring to conditions sufficient to substantially prevent the formation of embryoid bodies, is meant to encompass conditions under which the formation of embryoid bodies is suppressed. Under such conditions, embryoid bodies are formed from in some embodiments less than 10%, in some embodiments less than 5%, in some embodiments less than 3%, in some embodiments less than 1%, in some embodiments less than 0.1%, in some embodiments less than 0.01%, and in some embodiments less than 0.001% of the ES cells in the culture.

The term “target tissue” as used herein refers to an intended site for engraftment of a PEP following administration to a subject. In some embodiments of the disclosed methods, the target tissue is present within a vertebrate subject, in some embodiments a mammal, and in some embodiments a human. In some embodiments, the target tissue comprises a tissue of endodermal origin such as the liver, pancreas, spleen, or lung.

As used herein, the term “teratoma” refers to a tumor characterized by the unregulated development of ES cells. Teratomas often generate cells of several different cell and tissue types, such as of skin, hair, cartilage, and muscle. Mouse ES cells injected into syngeneic mice can form teratomas that exhibit disorganized differentiation, often with representatives of all three embryonic germ layers. Mouse ES cells combined into chimeras with normal preimplantation embryos and returned to the uterus participate in normal development.

As used herein, the phrases “treatment effective amount”, “therapeutically effective amount”, and “treatment amount” are used interchangeably and refer to an amount of a therapeutic composition (e.g., PEPs in a pharmaceutically acceptable carrier or excipient) sufficient to produce a measurable response (e.g., a biologically or clinically relevant response in a subject being treated). Actual dosage levels of PEPs in the pharmaceutical compositions of the presently disclosed subject matter can be varied so as to administer a sufficient number of PEPs so as to achieve the desired therapeutic response for a particular subject. The selected dosage level will depend upon several factors including, but not limited to the activity of the gene of interest in the target tissue, the route of administration, combination with other drugs or treatments, the severity of the condition being treated, and the condition and prior medical history of the subject being treated.

### III. Production of Putative Endoderm Precursors

### PEPs

The presently disclosed subject matter provides in some embodiments a method for generating a putative endoderm cell (PEP) from an embryonic stem (ES) cell. In some embodiments, the method comprises (a) culturing the embryonic stem cell in the absence of feeder cells and LIF; and (b) exposing the embryonic stem cell to acidic fibroblast growth factor in an amount and for a time sufficient to provide in some embodiments reduced expression of Oct4 and/or increased expression of an mRNA selected from the group consisting of α-fetoprotein, Gata4, albumin, FoxA2 (HNF-β3), and Sox17a in the embryonic stem cell, whereby a putative endoderm cell is generated. Additional markers of endodermal differentiation include, but are not limited to transthyretin (Tty), α 1-anti-trypsin, tyrosine aminotransferase, and glucose-6-phosphatase.

In some embodiments, the ES cell is a mammalian ES cells, for example, a human, mouse, rat, or pig ES cell, although the ES cell can be from any species. The isolation and maintenance of ES cells from various species are disclosed herein and in the following references, each of which is incorporated by reference in its entirety: Gerfen & Wheeler, 1995; Thomson et al., 1998; Ruhnke et al., 2003; and U.S. Pat. Nos. 6,194,635; 6,200,806; 6,271,436; and 6,875,607.

III.A. Establishment and Propagation of ES Cells

ES cells are undifferentiated, pluripotent cells derived in vitro from the inner cell mass of blastocyst stage preimplantation embryos (Evans et al., 1981; Martin, 1981). ES cells can maintain an undifferentiated state through serial passages using culturing techniques that are known in the art (see e.g., Robertson et al., 1987; Williams et al., 1988; Williams et al., 1988; Nagy et al., 1990; Nagy et al., 2003; Pera & Trounson, 2004). In some embodiments, ES cells are cultured on a fibroblast feeder layer and/or in the presence of Leukemia Inhibitory Factor (LIF) to maintain an undifferentiated state.

The cells of a feeder layer are typically mitotically inactivated with mitomycin C or gamma irradiation. An exemplary fibroblast cell that can be used to produce a feeder layer is the STO cell (available from American Type Culture Collection (ATCC), Manassas, Va., United States of America). Additionally, some feeder cells are available that have been modified to express LIF and/or a neomycin resistance gene (neo), the latter of which can be employed to grow ES cells and ES cell derivatives that have been transformed with an expression vector encoding a neomycin phosphotransferase (neo) coding sequence. In some embodiments, if a LIF-producing feeder cell is employed, the use of additional LIF in the ES cell propagation medium can be avoided. STO derivatives are available that have been modified to express both LIF and neo, such as the SNL76/7 fibroblast line described in McMahon & Bradley, 1990, and available from Dr. Allan Bradley, Baylor College of Medicine, Houston, Tex., United States of America. Other STO cell lines that have been modified to express both LIF and neo are available from Dr. Elizabeth Robertson of Harvard University, Cambridge, Mass., United States of America.

Alternatively, ES cells can be grown on a monolayer of murine embryonic fibroblasts (MEFs) that have been prepared as described in, for example, Loo & Costman, 1998. MEFs can also be prepared from a mouse embryo that has been genetically altered to express a selectable marker (see e.g., Tucker et al., 1997, describing a mouse that expresses resistance genes to G418, 6-thioguanine, puromycin, and hygromycin), which can aid in the propagation of ES cells and ES cell derivatives that have been transformed with recombinant vectors.

In some embodiments including, for example, when the ES cells are intended for use in producing PEPs for administration into humans, the presence of a feeder layer comprising cells from a species other than humans is disfavored. U.S. Pat. No. 6,800,480 discloses methods and materials for the growth of stem cells in a feeder-free culture. Thus, in some embodiments, the ES cells are maintained in culture in the absence of a feeder layer and maintained in an undifferentiated state by the addition of exogenous growth factors including, but not limited to LIF.

III.B. Generation of PEPs

The presently disclosed subject matter also provides a method for producing a reagent comprising an in vitro differentiated cell. In some embodiments, the method comprises (a) providing an embryonic stem (ES) cell or a pre-embryoid body cell; and (b) differentiating the ES cell or the pre-embryoid body cell in vitro to express a marker associated with a differentiated cell of interest. Optionally, the differentiated cell of interest normally expresses a gene of interest. As used herein, the phrase “differentiated cell of interest” refers to a cell from a target tissue into which a PEP is intended to differentiate in situ. In some embodiments, the differentiating comprises exposing the embryonic stem cell or the pre-embryoid body cell to a growth factor in an amount and for a time sufficient to cause the embryonic stem cell to reduce expression of Oct4 and increase expression of an endodermal marker selected from the group consisting of FoxA2 (Hnf-β3), Gata4, Sox17α, albumin, and α-fetoprotein. In some embodiments, the differentiating comprises growing the ES cells in the absence of feeder cells in an ES cell propagation medium for at least about 7 days, wherein the ES cell propagation medium contains about 100 ng/ml acidic FGF and is absent LIF.

Thus, in some embodiments, the presently disclosed subject matter provides a method for generating a putative endoderm cell (PEP) from an embryonic stem cell, the method comprising (a) culturing the embryonic stem cell in the absence of feeder cells and LIF; and (b) exposing the embryonic stem cell to acidic fibroblast growth factor in an amount and for a time sufficient to provide in some embodiments reduced expression of Oct4 and/or increased expression of an mRNA selected from the group consisting of a-fetoprotein, Gata4, albumin, FoxA2 (HNF-β3), albumin, and Sox 17a in the ES cell, whereby a putative endoderm cell is generated.

In order to prepare PEPs, undifferentiated ES cells are removed from culture conditions that promote maintenance of the undifferentiated state. In some embodiments, this involves removing the ES cells from the feeder layer and culturing the ES cells in media that does not contain LIF. Typically and as described hereinabove, ES cells grown under such conditions form embryoid bodies. However, unlike previous in vitro differentiation strategies (see e.g., Hamazaki et al., 2001; Chinzei et al., 2002; Yin et al., 2002), the formation of PEPs does not proceed through a stage wherein the ES cells are permitted to form embryoid bodies. Thus, in some embodiments the in vitro differentiation of ES cells to PEPs comprises preparing an essentially single-cell suspension of ES cells, which is then transferred to an appropriate growth substrate (e.g., a P35 collagen-coated tissue culture dish). The individual ES cells are then grown on the substrate in normal ES cell propagation medium in the absence of LIF but containing an inducing agent such as acidic fibroblast growth factor in an amount and for a time period sufficient to induce the differentiation of the ES cells into PEPs. As used herein, the term “ES cell propagation medium” refers to a medium that can be used to propagate ES cells in an undifferentiated state. An exemplary ES cell propagation medium comprises high glucose Dulbecco's modified Eagle's medium (DMEM) with 10-15% fetal bovine serum (or a serum free replacement product such as KNOCKOUT™ SR Serum Replacement for Embryonic Stem Cells, available from Invitrogen GIBCO® Corp., Carlsbad, Calif., United States of America), 2 mM glutamine, 0.1 mM β-mercaptoethanol, 100 units/ml penicillin, and 100 μg/ml streptomycin.

In some embodiments, the ES cells are differentiated in vitro by growing them in ES cell propagation medium supplemented with an inducing agent (e.g., a growth factor such as but not limited to acidic fibroblast growth factor) for at least about seven days. As the ES cells differentiate, the expression of various genes in the ES cells and their derivatives changes. Exemplary changes include the downregulation of certain gene products associated with the undifferentiated state and the concomitant upregulation of gene products expressed by endoderm derivatives. In some embodiments, as an ES cell differentiates in vitro into a PEP, the expression of Oct4 and/or nanog, genes associated with the undifferentiated state (Sylvester & Scholer, 1994; Chambers et al., 2003) decreases, and the expression of one or more of Gata4, FoxA2, Sox17α, Hex (also referred to as Hhex), albumin (Alb), and α-fetoprotein (Afp) increases. Additional markers of endodermal differentiation include, but are not limited to transthyretin (Tty), α 1-anti-trypsin, tyrosine aminotransferase, and glucose-6-phosphatase.

Thus, the changes in expression of these genes can be monitored to assess the state of the ES-derived cell's differentiation. Nucleotide and amino acid sequences for these genes and gene products from various species are present in the GENBANK® database, representatives of which can be found under the Accession Numbers outlined in Table 1.

TABLE 1
Exemplary GENBANK ® Accession Numbers

| | Gene | | | |
|---|---|---|---|---|
| | Human | | Mouse | |
| | Nucleotide | Amino Acid | Nucleotide | Amino Acid |
| | Sequence | Sequence | Sequence | Sequence |
| | Accession | Accession | Accession | Accession |
| | Number(s) | Number(s) | Number(s) | Number(s) |
| Afp | NM_001134 | NP_001125 | NM_007423 | NP_031449 |
| Alb | NM_000477 | NP_000468 | NM_009654 | NP_033784 |
| FoxA2 | NM_021784; | NP_068556; | NM_010446 | NM_034576 |
| | NM_153675 | NP_710141 | | |
| Gata4 | NM_002052 | NP_002043 | NM_008092 | NP_032118 |
| Hex | NM_002729 | NP_002720 | NM_008245 | NP_032271 |
| Oct4 | NM_002701 | NP_002692 | NM_013633 | NP_038661 |
| Sox17α | NM_022454 | NP_071899 | NM_011441 | NP_035571 |
| Tty | NM_000371 | NP_000362 | NM_013697 | NP_038725 |

The GENBANK® database includes additional nucleotide and amino acid sequences for these genes and gene products from other species including, for example, chimp, rhesus macaque, gorilla, pig, cow, horse, dog, cat, rat, chicken, Xenopus, and Medaka.

Additionally, the modulation of the expression of the genes listed above, or indeed of any gene that is differentially expressed in an ES cell versus a PEP, can also be used to purify undifferentiated ES cells away from the PEPs prior to the introduction of the PEPs into a subject. Methods of distinguishing and/or sorting cells based on differential gene expression are known in the art, and include, but are not limited to fluorescence activated cell sorting (FACS). While applicants do not wish to be bound by any particular theory of operation, purifying PEPs away from ES cells and other undifferentiated cells can decrease the incidence of teratoma formation that can result from the administration of ES cell derivatives to subjects.

### IV. Methods of Treating Disease

The presently disclosed subject matter provides in some embodiments methods for treating a disease associated with abnormal or missing function of a tissue. As used herein, the phrase “disease associated with abnormal or missing function of a tissue” refers to a disease at least one symptom or consequence of which results from an abnormal or missing biological activity in at least one tissue or cell type in the subject. Thus, the phrase “disease associated with abnormal or missing function of a tissue” refers to genetic diseases wherein one or more gene products are absent or have abnormal activity (for example, activity that is higher than, lower than, or otherwise different from that seen in subjects that do not have the disease, such that the abnormal activity results in one or more symptoms or consequences to the subject) as well as diseases that result from other abnormal or missing functions of at least one tissue or cell type in the subject. A non-limiting example of the latter situation would be where a tissue or cell type was either missing nor functioned abnormally as a result of trauma, multiple genetic defects, autoimmune disease, cancer, inflammation, or any other cause not directly attributed to a discrete genetic defect. It is understood, however, that discrete genetic defects can result in tissues and cell types being absent or abnormal in a subject, and as such, these examples of diseases associated with abnormal or missing function of a tissue are not mutually exclusive.

Representative tissues include but are not limited to endoderm-derived tissues. The presently disclosed methods can comprise providing a putative endoderm cell (PEP); and introducing into the subject the PEP, whereby the PEP becomes engrafted (for example, stably engrafted) in an endoderm-derived tissue of the subject and provides a normal function to or replaces the missing function in the subject.

In some embodiments, providing the PEP can comprise differentiating an embryonic stem cell or a pre-embryoid body cell in vitro to form a PEP. Also, representative endoderm-derived tissues include but are not limited to thyroid gland, liver, intestine, pancreas, spleen, and lung.

IV.A. Gene-Based Diseases

The presently disclosed subject matter provides a method for treating a disease. In some embodiments, the disease is a disease associated with abnormal or absent expression of a gene of interest in a subject, the method comprising (a) differentiating an embryonic stem cell or a pre-embryoid body cell comprising the gene of interest in vitro to form a putative endoderm cell (PEP); and (b) introducing into the subject the PEP, whereby the PEP becomes stably engrafted in an endoderm-derived tissue of the subject and expresses the gene of interest.

As used herein, the phrases “disease associated with abnormal or absent expression of a gene of interest” and “disease associated with abnormal or absent gene expression” are used interchangeably and refer to any disease a symptom of which results from the expression (or lack of expression) of any gene in any cell type of the subject that is different from that normally seen in the same cell type of an unaffected (i.e., normal) subject of the same species. Diseases associated with abnormal or absent expression of a gene of interest are typically diseases that result from absent or insufficient production of the product of a gene of interest (sometimes referred to as genetic diseases or gene-based diseases), although the presently disclosed subject matter is not limited to only these circumstances.

Diseases that are associated with abnormal or absent gene expression would be apparent to one of ordinary skill in the art after a review of the present disclosure. An exemplary, non-limiting disease associated with abnormal or absent gene expression is hemophilia B, which is characterized by severe bleeding diathesis caused by the absence of the Factor IX protein. Other exemplary diseases include, but are not limited to familial hypercholesterolemia, factor VIII deficiency (Hemophilia A), Methionine Adenosyltransferase (MAT1A) Deficiency, Phenylalanine Hydroxylase Deficiency (phenylketonuria), Alpha-1-Antitrypsin Deficiency, and the various glycogen storage diseases. Listings of exemplary diseases that can be treated with the presently disclosed compositions and methods are presented in OMIM™—ONLINE MENDELIAN INHERITANCE IN MAN™, available from the website of the National Center for Biotechnology Information (NCBI) and in Wilson et al., 1991.

Thus, in some embodiments, a disease associated with abnormal or absent expression of a gene of interest results from a mutation in a gene in a cell that is present in an otherwise normal cell or tissue type in a subject. In some embodiments, however, a disease associated with abnormal or absent expression of a gene of interest results not from a mutation in a gene of interest in a cell or tissue type, but from an absence of normal function of the cell or tissue type in the subject. The absence of normal function can result, for example, from an absence (either absolute or relative to a normal subject) of the cell or tissue type in the subject, or a defect in the normal functioning of the cell or tissue type in the subject that is not associated with a single gene defect. Such diseases associated with abnormal or absence

As used herein, the phrase “gene of interest” refers to any gene for which altering the expression level of which would result in a therapeutically beneficial outcome for the subject. As discussed hereinabove, this treatment would typically involve providing a PEP to a subject that does not produce the gene product (or does not produce sufficient quantities of the gene product in relevant cell types) resulting in the production of sufficient amounts of a gene product encoded by the genome of the PEP (or its progeny) in order to ameliorate at least one symptom of the disease. In some embodiments, this is accomplished by differentiating ES cells or other pluripotent or totipotent cells (e.g., stem cells) in vitro to produce cells that express the gene of interest either as a direct result of the in vitro differentiating event or as a result of being placed within a tissue environment in the subject (e.g., engraftment into an endoderm-derived tissue of the subject such as the liver) that induces the cell to produce the product of the gene of interest.

As used herein, the phrase “stably engrafted” refers to engraftment of a target tissue with a PEP that is characterized by a relatively constant number of PEPs and their progeny being present in the target organ over a period of time once an equilibrium is established. For example, it is understood that not every administered PEPs will engraft the target tissue, but after those that do not engraft are removed from the target tissue via natural processes, an equilibrium is established wherein the number of engrafted PEPs (and/or their progeny) does not decrease substantially over a given time period. Thus, a target tissue is stably engrafted if the number of PEPs and/or their progeny does not substantially decline for, in some embodiments at least about two weeks, in some embodiments at least about four weeks, in some embodiments at least about six weeks, in some embodiments at least about eight weeks, in some embodiments at least about ten weeks, in some embodiments at least about twelve weeks, in some embodiments at least about three months, in some embodiments at least about four months, in some embodiments at least about five months, in some embodiments at least about six months, in some embodiments at least about twelve months, in some embodiments at least about eighteen months, and in some embodiments at least about two years.

As used herein, the term “endoderm-derived tissue” refers to a tissue or organ that is primarily derived from endoderm. Representative endoderm-derived tissues include thyroid gland, liver, intestine, pancreas, spleen, and lung. In some embodiments, an endoderm-derived tissue is selected from the group consisting of liver and pancreas, and in some embodiments an endoderm-derived tissue is liver.

As disclosed herein, embryonic stem (ES) cells are differentiated in vitro to generate putative endodermal precursors (PEPs), which can then be administered to subjects in order to treat diseases associated with abnormal or missing function of a tissue (e.g., abnormal or absent expression of a gene of interest) in the subject. In some embodiments, the ES cell is from the same species as the subject to be treated. In some embodiments, the ES cell is syngeneic to the subject, and in some embodiments the ES cell is allogeneic to the subject.

As disclosed herein, the PEPs of the presently disclosed subject matter can engraft into a subject without inducing an anti-PEP immune response in the subject (i.e., without initiating a host-versus-graft reaction). Thus, long-term engraftment can occur across histocompatability barriers without the requirement for the use of immunosuppressants. In some embodiments, however, the subject can be treated as necessary with immunosuppressant drugs such as cyclosporin, azathioprines, or corticosteroids using well-known techniques. Representative immunosuppressive drugs also include basiliximab (SIMULECT®; available from Novartis Pharmaceuticals Corp., East Hanover, N.J., United States of America), daclizumab (ZENAPAX®, available from Hoffmann-La Roche Inc., Nutley, N.J., United States of America), muromonab CD3 (ORTHOCLONE OKT3®, available from Ortho Biotech Products, L.P., Bridgewater, N.J., United States of America) and tacrolimus (PROGRAF®, available from Astellas Pharma US, Inc., Deerfield, Ill., United States of America).

As used herein, the phrase “long-term engraftment”, and grammatical variants thereof, refers to a stability of engraftment that exceeds a minimum duration of, in some embodiments 10 days, in some embodiments 30 days, in some embodiments 60 days, in some embodiments 90 days, in some embodiments 120, in some embodiments 150, in some embodiments 180 days, in some embodiments 210, in some embodiments 240, in some embodiments 270, and in some embodiments 300 days. It is to be understood that in order for the engraftment of PEPs to be “long-term”, it is not necessary that the administered PEPs themselves persist in the target tissue for 10, 30, 60, 90, 120, 150, 180, 210, 240, 270, 300, or more days. Rather, long-term engraftment can be accomplished when the progeny of the administered PEPs persist for at least 10, 30, 60, 90, 120, 150, 180, 210, 240, 270, 300, or more days.

In some embodiments, the ES cells that are used to generate PEPs are wild type ES cells, meaning that the ES cells have not been modified by the introduction of exogenous nucleic acids that are intended to be expressed in the subject after in vitro differentiation into PEPs and administration to the subject. As used herein, a “wild type ES cell” can carry some exogenous (for example, recombinant) nucleotide sequences, with the proviso that the exogenous nucleotide sequences neither encode the gene of interest nor directly modulate the expression of the gene of interest in the PEP. Exemplary exogenous nucleotide sequences that can be present within wild type ES cells include, but are not limited to reporter genes (including, but not limited to a coding sequence encoding GFP) and selective marker genes (including, but not limited to neo).

When wild type ES cells are differentiated in vitro to form PEPs, the wild type ES cells give rise to wild type PEPs, which refers to PEPs that after administration into a subject will express an endogenous gene of interest that is regulated only by its naturally occurring, endogenous regulatory elements. Thus, in some embodiments, the gene of interest is normally expressed in the tissue into which the PEPs are administered and become engrafted (e.g., the liver).

Thus, wild type PEPs can be used to treat diseases that are characterized by abnormal or missing function of a tissue in a subject, such as can occur, for example, from abnormal or absent expression of a gene of interest in the tissue into which the PEPs are introduced. For example, diseases characterized by mutations in genes that are expressed in the liver can be treated by administering wild type PEPs to the liver of a subject with the disease, which after engraftment would be expected to express the gene in the liver.

Thus, in some embodiments the PEPs are intended to express an endogenous gene in the tissue into which they are introduced. In some embodiments, the PEPs are administered into the liver parenchyma, where they become engrafted.

In some embodiments, however, the PEPs are intended to express a gene that is not normally expressed in the recipient tissue, and in order to accomplish this, the genomes of the ES cells are manipulated to contain exogenous sequences (i.e., transgenes, exogenous regulatory elements, and/or combinations thereof). Accordingly, in some embodiments the in vitro differentiated PEP further comprises a coding sequence that is not normally expressed in the target tissue of the subject in the absence of disease, but is normally expressed in another tissue or cell type of the subject in the absence of disease.

Conventional gene transfer methods can be used to introduce DNA into ES cells. The precise method used to introduce a replacement gene (e.g., a clotting factor or metabolic protein) is not to be considered a limitation of the presently disclosed subject matter. For example, physical methods for the introduction of DNA into ES cells include microinjection and electroporation. Chemical methods such as co-precipitation with calcium phosphate and incorporation of DNA into liposomes are also standard methods of introducing DNA into mammalian cells. DNA is introduced using standard vectors, such as those derived from murine and avian retroviruses (see e.g., Gluzman et al., 1988). Standard recombinant DNA methods are well known in the art (see e.g., Ausubel et al., 1989), and viral vectors for gene therapy have been developed and successfully used clinically (see e.g., Rosenberg et al., 1990).

Typically, ES cell transformation involves electroporation of the ES cells (see e.g., Thomas & Capecchi, 1987) with a construct comprising a coding sequence(s) or regulatory sequence(s) of interest. In some embodiments, a plurality of ES cells is electroporated with an expression construct encoding a gene of interest operatively linked to a promoter that would be active in the target tissue (e.g., the liver). In some embodiments, the ES cell is co-transformed with a first expression construct encoding the gene of interest and with a second expression construct encoding a selectable marker (e.g., neo), which allows for the positive selection of ES cells that have been co-transformed with the first and second expression constructs by growing the ES cells in the presence of selective medium (e.g. medium containing the drug G418). Colonies of ES cells that survive the selection can then be screened for the presence of the gene of interest using standard molecular biology techniques such as PCR, Southern blotting, etc. (see e.g., Sambrook & Russell, 2000).

In some embodiments, a promoter that is active in the target tissue (for example, a target-tissue specific promoter or a constitutive promoter) is “knocked in” to an endogenous gene locus to “turn on” the endogenous gene in the target tissue. Methods for performing knock in targeting are also known in the art (see e.g., Jin et al., 2000).

Thus, in some embodiments a PEP is derived from an ES cell the genome of which comprises a transgene encoding a gene of interest operatively linked to a promoter. Any transgene can be used to transform ES cells in order to produce genes of interest in target tissues of a subject, with the proviso that in order to achieve expression of the transgene in the target tissue, the promoter operatively linked to the transgene should be active in the engrafted PEPs and/or in their progeny. After a review of the present disclosure, one of ordinary skill in the art would understand which promoters and/or regulatory elements can be chosen based on factors such as what the target tissue is, whether higher or lower expression of the transgene is desirable, whether the expression of the transgene should optimally be inducible, etc.

IV.B. Other Diseases

In some embodiments, the presently disclosed subject matter provides a method of treating a disease that is associated with absent or abnormal function of a tissue or cell type. A representative, non-limiting example of such a disease is diabetes, in which glucose regulation is adversely affected by absent or abnormal function of pancreatic β-cells. In some embodiments, a disease such as diabetes is treated by administering a PEP to a subject in order to provide the function that is abnormal or absent in the subject.

In some embodiments, the PEP has been modified to contain a transgene that provides the absent or abnormal function by causing the PEP to differentiate into the desired cell type. One particular example of a transgene that can be used to direct engrafted PEPs to function as insulin-producing cells is the pancreatic and duodenal homeobox gene 1 (Pdx-1) described in Sapir et al., 2000 (see also Ber et al., 2003; Horb et al., 2003; Ber et al., 2003; Kojima et al., 2003; Ber et al., 2003; Meivar-Levy et al., 2003; Miyatsuka et al., 2003, and U.S. Pat. No. 6,774,120 to Ferber, the contents of which are incorporated herein by reference in their entireties). Ectopic expression of the Pdx-1 gene results in the transdifferentiation of liver cells to pancreas-like cells that produce, store, and release insulin in a glucose-regulated manner. Thus, by creating ES cells that express Pdx-1 (for example, under a constitutive or target tissue-specific promoter), differentiating them in vitro to form PEPs, and transferring the PEPs into a target tissue (for example, the liver), it is possible to ameliorate the symptoms and consequences of abnormal insulin production (such as, for example, diabetes) in a subject.

In some embodiments, the disease that is associated with absent or abnormal function of a tissue or cell type results from undesirable cell death or lack of cell growth and/or regeneration. Non-limiting examples of such a disease include cirrhosis of the liver and cancer of an endoderm-derived tissue, such as liver cancer. As cirrhosis can be caused by the death of liver cells without adequate regeneration of new liver cells, the administration of PEPs to a subject can overcome the deficit in liver cells.

IV.C. Administration of PEPs to Subjects

For analysis and/or transplantation into a subject, the PEPs can be treated with trypsin/EDTA in order to form a single cell suspension and resuspended in an appropriate pharmaceutically acceptable carrier such as phosphate-buffered saline.

IV.C.1. Subjects

The subjects treated in the presently disclosed subject matter are in some embodiments human subjects, although it is to be understood that the presently disclosed subject matter is effective with respect to all vertebrate animals, including mammals, which are intended to be included in the term “subject”. Moreover, a mammal is understood to include any mammalian species in which treatment or prevention of a disease is desirable, particularly agricultural and domestic mammalian species.

More particularly provided is the treatment of mammals such as humans, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, for example, poultry, such as turkeys, chickens, ducks, geese, guinea fowl, and the like, as they are also of economic importance to humans. Thus, contemplated is the treatment of livestock, including, but not limited to, domesticated swine (pigs and hogs), ruminants, horses, poultry, and the like.

IV.C.2. Formulation

The cells of the presently disclosed subject matter comprise in some embodiments a composition that includes a carrier, particularly a pharmaceutically acceptable carrier. Any suitable pharmaceutical formulation can be used to prepare the compositions for administration to a subject.

For example, suitable formulations can include aqueous and non-aqueous sterile injection solutions that can contain anti-oxidants, buffers, bacteriostatics, bactericidal antibiotics and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are SDS, in one example in the range of 0.1 to 10 mg/ml, in another example about 2.0 mg/ml; and/or mannitol or another sugar, for example in the range of 10 to 100 mg/ml, in another example about 30 mg/ml; and/or phosphate-buffered saline (PBS).

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

The therapeutic regimens and compositions of the presently disclosed subject matter can be used with additional adjuvants or biological response modifiers including, but not limited to, cytokines and other immunomodulating compounds.

IV.C.3. Administration

Administration of the cells of the presently disclosed subject matter can be by any method known to one of ordinary skill in the art. In some embodiments, suitable methods for administration of the cells of the presently disclosed subject matter include, but are not limited to injection into the parenchyma of the target tissue (e.g., the liver parenchyma). For example, when injected into the liver, PEPs access the vascular space, pass into the sinusoid, and traverse the fensetra of the endothelial cells. They are viable in the “space of Disse”. A blanching that can be seen under low magnification confirms the displacement of blood with cell-containing media within the parenchyma. In some embodiments, a subject can receive a partial hepatectomy prior to the administration of the PEPs.

IV.C.4. Dose

An effective dose of a composition of the presently disclosed subject matter is administered to a subject in need thereof. A “treatment effective amount” or a “therapeutic amount” is an amount of a therapeutic composition (e.g., PEPs in a pharmaceutically acceptable carrier or excipient) sufficient to produce a biologically or clinically relevant response in a subject being treated. The actual number of PEPs in the compositions of the presently disclosed subject matter can be varied so as to administer a number of the PEPs that is effective to achieve the desired therapeutic response for a particular subject.

The potency of a composition can vary, and therefore a “treatment effective amount” can vary. However, using standard assay methods, one skilled in the art can readily assess the potency and efficacy of a PEP of the presently disclosed subject matter, and adjust the therapeutic regimen accordingly.

After review of the disclosure of the presently disclosed subject matter presented herein, one of ordinary skill in the art can tailor the dosages to an individual subject, taking into account the particular formulation, method of administration to be used with the composition, and particular disease treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

### V. Other Applications

In some embodiments, the presently disclosed subject matter also provides a method of investigating differentiation of a tissue or cell type in vivo or in vitro. In some embodiments, the method comprises administering a PEP into a tissue of a subject, and monitoring the differentiation of the PEP in the tissue. In some embodiments, the PEP further comprises a transgene encoding a detectable marker, facilitating the differentiation of the PEP in the tissue.

In some embodiments, the transgene encoding the detectable marker is operatively linked to a promoter that is transcriptionally active in the tissue at one or more stages of differentiation of a cell normally found in the tissue.

In this way, the PEP can be employed for investigating the differentiation of cells normally found in the tissue, as well as being employed for screening for modulators of such differentiation.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

### Culture and Differentiation of ES Cells

Enhanced GFP-Expressing ES Cells. To facilitate the identification of successful engraftment, PEPs were differentiated from a murine ES cell line that constitutively expressed a single-copy transgene encoding an enhanced GFP (Stratagene, La Jolla, Calif., United States of America) operatively linked to a ubiquitously expressed β-actin promoter (Fair et al., 2003). The encoded GFP localized to the nucleus. This ES cell line was derived from a strain 129P2/Ola mouse. Cells were maintained on murine embryonic fibroblast (MEF) feeder layers that produced leukemic inhibitory factor (LIF). To ensure viability and pluripotency, ES cell medium was changed daily, and ES cells were passaged every 2-3 days. The karyotypes of representative ES cells were checked at intervals to ensure euploidy.

ES Cell Culture. ES cell propagation medium consisted of high glucose DMEM (Sigma Chemical Co., St. Louis, Mo., United States of America) supplemented with 15% FBS (Sigma), 0.1 mM, β-mercaptoethanol (Sigma), 100 units/ml penicillin (Sigma), and 100 μg/ml streptomycin (Invitrogen GIBCO®, Carlsbad, Calif., United States of America).

ES Cell Differentiation. In order to directly differentiate ES cells in vitro, the cells were removed from their feeders, plated onto P35 collagen-coated plates, and grown in ES cell differentiation medium. ES cell differentiation medium was ES cell propagation medium supplemented with 100 ng/ml acidic FGF (Sigma). The medium was carefully changed daily, and the cells were allowed to obtain an approximately 50% density. When the cells were ready for transplantation or analysis, they were removed from the plates with 0.05% trypsin/0.02% tetrasodium EDTA in PBS, counted, and stained for viability with Trypan blue.

### Example 2

### F-IX ELISA

An ELISA specific for F-IX was performed as described by Gui et al., 2002, with the following modifications. Both the capture and detection antibodies were sheep anti-mouse specific for F-IX (a kind gift of Affinity Biologicals, Ancaster, Ontario, Canada) with the detection antibody covalently linked to horseradish peroxidase. Purified mouse F-IX (produced and purified in the laboratory of D. Stafford, University of North Carolina, Chapel Hill, N.C., United States of America) was used as the standard.

### Example 3

### Quantitative RT-PCR

Real-time fluorescent quantitative PCR was performed to assess the expression of mouse Oct4, FoxA2, Gata4, Sox17α, Afp, and Alb using an ABI PRISM® 7700 (Applied Biosystems, Foster City, Calif., United States of America). The sequences of the oligonucleotides used for each quantitation are listed in Table 2 below.

TABLE 2

Oligonucleotides used for Quantitative
RT-PCR

| | | |
|---|---|---|
| Gene | Sequence (5′ to 3′) | SEQ ID NO. |

| | | |
|---|---|---|
| Oct4 | | |
| Forward | GTTTGCCAAGCTGCTGAAGC | 1 |
| Reverse | GAAGCGACAGATGGTGGTCT | 2 |
| Fluoroprobe | CTCACCCTGGGCGTTCTCTTTGGA | 3 |
| FoxA2 | | |
| Forward | CAAGGCCTACGAACAGGTCATG | 4 |
| Reverse | CTCCTTGGTAGTAGGAAGTGTCTGCA | 5 |
| Fluoroprobe | AGCCTGGATGCCTCGCCCCT | 6 |
| Gata4 | | |
| Forward | GTAGGCCTCTCCTGTGCCAA | 7 |
| Reverse | TACATACAGGCTCACCCTCG | 8 |
| Fluoroprobe | TGCCAGACTACCACCACCACGCTG | 9 |
| Sox17α | | |
| Forward | GGCCGATGAACGCCTTT | 10 |
| Reverse | TCTGGGTTCTGCTGTGCCA | 11 |
| Fluoroprobe | CTTGCGTTCGTCTTTGGCCCACA | 12 |
| Afp | | |
| Forward | ATTGCCTCCACGTGCTGCCA | 13 |
| Reverse | GAAAATGTCGGCCATTCCCT | 14 |
| Fluoroprobe | CAGCCGGACCATTTCTCCTCGCT | 15 |
| Alb | | |
| Forward | GGCACCAAGTGTTGTACACT | 16 |
| Reverse | AGCAGACACACACGGTTCAG | 17 |
| Fluoroprobe | CCTTGTGTGGAGGACTATCTGTCTGC | 18 |

### Example 4

### Fluorescent Stereomicroscopy

To initially identify the engraftment of PEPs, fresh liver explants were examined with a stereomicroscope (MZ16FA, Leica Microsystems, Wetzlar, Germany) using a GFP2 long-pass filter (100447084, Leica Microsystems) to detect the presence of GFP-positive cells.

Procedure Modifications for F-IX-Deficient Mice. The F-IX knockout mice used in the disclosed transplant studies were first confirmed to be F-IX-negative by PCR genotyping, and a day 0 F-IX baseline was collected. To perform the partial hepatectomy, the mice were given 100 units/kg recombinant human F-IX (Ahmad et al., 1995), both by intramuscular (i.m.) and subcutaneous (s.c.) injection after anesthesia, and another s.c. dose 4 hours after the operation. At the time when the GFP-PEP injections were performed (on postoperative day 3), the i.m. and s.c. doses of human F-IX were repeated. F-IX levels were assessed by ELISA on the plasma (Gui et al., 2002) 4, 7, and 10 days after injection and on a weekly basis thereafter. Recombinant human F-IX (stock concentration; 575 μg/ml) was from the laboratory of Darrel W. Stafford (University of North Carolina, Chapel Hill, N.C., United States of America).

Partial Hepatectomy. Adult mice (C.129P2F9^{tm1Dws}/Fre, B6.129P2^{tm1Dws}/Fre) [F-IX, BALB/c and C57BL/6] 6-15 weeks of age were obtained from the laboratory of Darrel W. Stafford (University of North Carolina, Chapel Hill, N.C., United States of America). Wild type BALB/c and C57BL/6 mice were obtained from The Jackson Laboratory (Bar Harbor, Me., United States of America). Mice were kept in a Department of Laboratory Animal Medicine facility at the University of North Carolina at Chapel Hill, and were watered and fed regular chow ad libitum. Mice were anesthetized with an s.c. injection of ketamine/xylazene mixture (60 mg/kg ketamine and 6 mg/kg xylazene). Once under anesthesia, the thoracic and abdominal surfaces were shaved using appropriate animal clippers (Oster Golden A5), and the shaved area was decontaminated by applying BETADINE®, followed by 70% ethanol. Once the mouse was anesthetized, the abdominal cavity was entered through a 2 cm incision starting just below the sternum and traveling straight down the abdominal wall. The median liver lobes (left and right) and the large left lateral lobe were isolated, tied off with 3-0 ties, and removed. After removing the lobes, the abdomen was closed with 4-0 prolene or silk sutures.

Before the abdominal wall was closed, 1 ml of room temperature PBS was placed in the abdomen. Mice then recovered in a clean, dry, warm cage under a warming lamp until they were mobile. Mice were given a 0.1 g/kg i.p. dose of buprenorphine every 12 hours after surgery for 2 days and were monitored closely thereafter.

Hepatic Injection of ES-Derived Cells. PEPs (1×10⁶) were suspended in 100 μl of PBS with 1% FBS and kept at 4° C. for about 30 minutes. Recipient mice were anesthetized with an s.c. injection of ketamine/xylazene mixture (60 mg/kg ketamine and 6 mg/kg xylazene) using a 28 or 31 gauge syringe. Once under anesthesia, a small incision was created below the right costal margin, and the liver lobe was delivered into view with a cotton swab. The cell suspension was injected directly into the parenchyma. Postoperative pain management was performed as above.

Tissue Processing. To further characterize cell engraftment, treated mice that showed significant levels of F-IX by ELISA were anesthetized with 60 mg/kg ketamine and 6 mg/kg xylazene and perfused transcardially with PBS, followed by 4% paraformaldehyde. The liver was removed and fixed overnight in 4% paraformaldehyde in PBS, transferred to 30% sucrose in PBS overnight, and then mounted in OCT™ (Optimal Cutting Temperature) Compound (TISSUE-TEK®, Sakura Finetek U.S.A., Inc., Torrance, Calif., United States of America, no. 4583) for quick-freezing. The liver samples were sliced into 10 μm thick sections with a cryostat (Leica Microsystems, Bannockburn, Ill., United States of America), mounted onto clean SUPERFROST™ Plus slides, air-dried, and stored at −80° C. until processed. Tissue samples from positive (wild types) and negative (non-transplanted F-IX^{-/-}) samples were obtained and processed identically to the experimental samples.

Histology. Samples were fixed in 4% paraformaldehyde overnight and transferred into 30% sucrose for a minimum of 24 hours. Samples were then placed in a cryomold (TISSUE-TEK®, 25×20×5 mm) with OCT™ Compound (TISSUE-TEK®), frozen at −80° for a minimum of 24 hours, and processed by cryosectioning in Leica CM 3050 S cryostat. Cryosections of 10 to 12 μm thickness were rehydrated in PBS, mounted with aqueous mounting medium (DakoCytomation California Inc., Carpinteria, Calif., United States of America), and examined by fluorescence microscopy for the presence of GFP cells.

Immunofluorescence. Slides with tissue cryosections processed as above were washed twice in PBS, permeabilized, and blocked against nonspecific binding in blocking buffer (5% normal serum/2% bovine serum albumin (BSA)/0.1% Triton X-100 in phosphate-buffered saline (PBS)) for 30 minutes. Samples were then incubated with sheep anti-rat F-IX antibody (Haematologic Technologies, Essex Junction, Vt., United States of America) for 90 minutes at room temperature at a final dilution of 1:200 in blocking buffer. After the incubation, samples were washed three times with PBS/0.05% Triton X-100, followed by incubation with Texas Red-labeled anti-sheep Ig (Fab′2) IgG (Jackson ImmunoResearch Laboratories, West Grove, Pa., United States of America) diluted 1:200 in blocking buffer for 30 minutes. After three washings in PBS/0.1% Triton X-100, samples were incubated with a rabbit polyclonal anti-GFP antibody conjugated to Alexa Fluor 488 (Invitrogen MOLECULAR PROBES™, Carlsbad, Calif., United States of America) for 1 hour at room temperature (1:400 dilution in blocking buffer) after three washings with PBS/0.1% Triton X-100 and mounted with VECTASHIELD® mounting media (Vector Laboratories, Burlingame, Calif., United States of America). Processed slides were examined under a Zeiss Axiovert S100 fluorescent microscope (Carl Zeiss, Inc., Thornwood, N.Y., United States of America), and representative slides were further analyzed by confocal laser-scanning microscopy performed on a Zeiss LSM5 Pascal microscope (Carl Zeiss, Inc.).

Laser-Capture Microdissection (LCM). Slides containing frozen sections were placed on the stage of an Arcturus PIXCELL® II LCM system (Arcturus Bioscience, Inc., Mountain View, Calif., United States of America). Either a standard CAPSURE® or a new CAPSURE® HS LCM cap (Arcturus Bioscience, Inc.) was placed on the section. Desired areas of tissue were maneuvered under the target beam, and the IR laser was fired to melt the special plastic membrane from the cap into the tissue. After all desired areas were shot in this way, the cap was lifted off the tissue, bringing with it only the selected tissue. Fragments of loose tissue were removed from the cap by pressing the cap onto the adhesive surface of a new POSTIT® (3M, St. Paul, Minn., United States of America) note. The cap was then placed into an Eppendorf tube containing extraction buffer. Images were made before capture, after capture, and of the cap containing captured material.

Antidonor Antibody Testing. Serum from graft recipient mice was tested for antibodies reactive to strain 129 spleen cells because PEPs were derived from strain 129 and share MHC with 129 splenocytes. Serum from BALB/c mice engrafted with PEPs was diluted and incubated with 129 target cells. Cells were subsequently washed and incubated with goat anti-mouse Ig. Cells were washed and run on a flow cytometer (FACSCalibur, BD Biosciences, Mountain View, Calif., United States of America), and the data were analyzed by using SUMMIT software (Cytomation, Fort Collins, Colo., United States of America). A polyclonal antiserum, (AXB10.D2), F1 anti-B10.A (5R), was used as a positive control.

### Example 5

### Directed In Vitro Differentiation of ES Cells

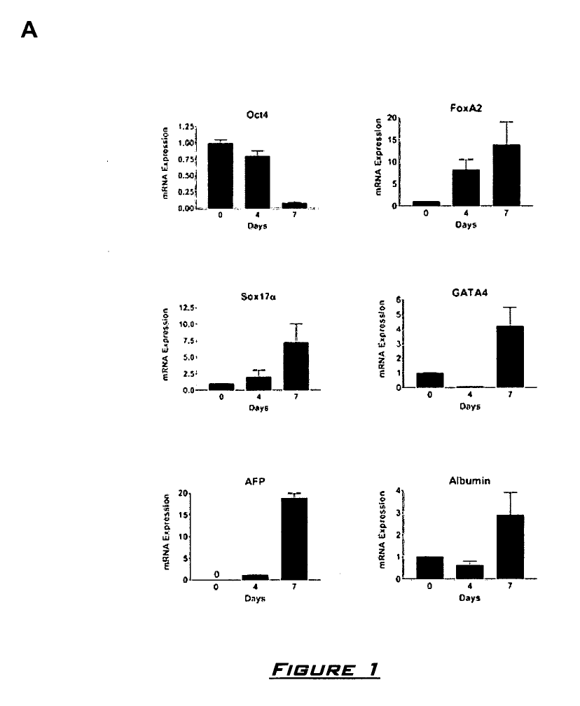

ES cells, removed from their feeder layer, were stimulated with acidic FGF (aFGF) at 100 ng/ml for 7 days. As shown in FIG. 1A, Quantitative RT-PCR (Kim et al, 2002) indicated that the cells displayed a statistically significant reduction in mRNA of Oct4 (p<0.01), and an increase in mRNA of Afp (p<0.01), Gata4 (p<0.05), Alb, (p<0.05), FoxA2 (p=0.06), and Sox17α (p=0.06). These molecular changes coincide with dramatic morphologic changes and the appearance of large cells with distinctive growth patterns (see FIG. 1B). The resulting cells are referred to as PEPs.

### Example 6

### Cellular Transplantation of PEPs

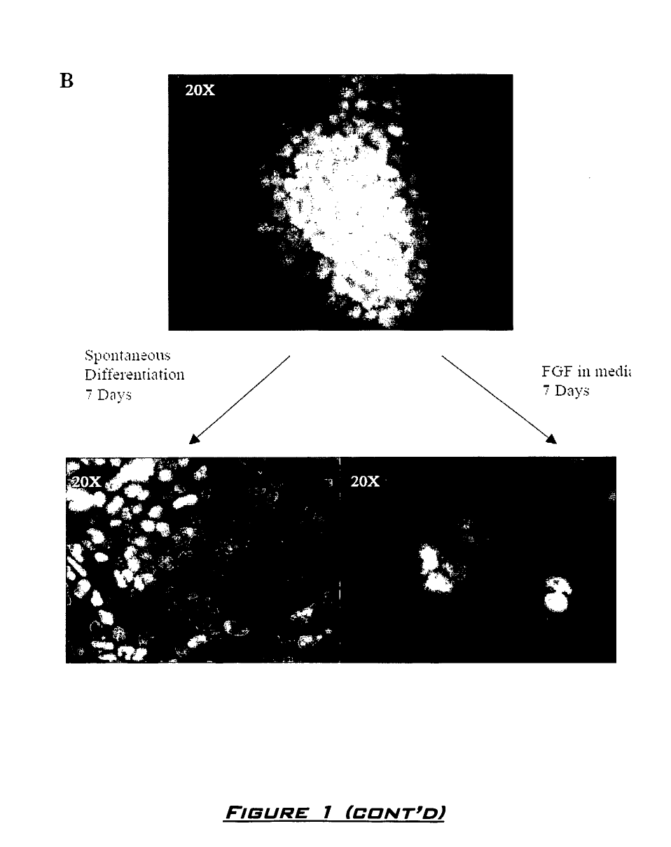

To optimize the potential conditions for PEPs to engraft, partially hepatectomies were initially performed on the recipient mice. Numerous studies have explored possible routes of delivering cells for engraftment in the hepatic parenchyma (Gupta et al., 1999). Preliminary injection studies were performed that showed that direct injection of PEPs into the liver resulted in their dispersal throughout the parenchymal vasculature. This method was chosen as providing a direct delivery of PEPs to the intended engraftment location. When fresh explanted liver tissue was screened by 3D fluorescent microscopy, the exogenous GFP-ES-derived cells were easily visualized and distinguished from the endogenous hepatic parenchyma, which autofluoresced, allowing for immediate and accurate detection of engraftment. FIGS. 2B and 2C illustrate typical results 20 days after injecting strain 129 (H2^{b})-derived PEPs into a C57BL/6 recipient (also H2^{b}). The GFP-PEP hepatic engraftment displayed a consistent pattern with the GFP-PEP cells coursing along the sinusoids within the space of Disse. Engraftment comparable to that shown in FIGS. 2B and 2C occurred in 10 of 19 mice (52%) injected with PEPs versus 0 of 14 mice injected with 1×10⁶ undifferentiated ES cells (X²=10.6; P<0.01).

### Example 7

### PEP Engraftment of Allogeneic Liver

Having observed robust engraftment in a MHC type (H2^{b}) recipient, whether GFP-PEP could be transplanted across a major MHC barrier was investigated. Using the same in vitro and in vivo protocols as above, partially hepatectomized BALB/c (H2^{d}) mice were employed, which were strain- and MHC-mismatched to the cells from which the PEPs (strain 129; H2^{b}) were derived. The PEPs were then injected as described hereinabove, and this injection resulted in engraftment in 5 of 10 (50%) of the recipients 10 days after the GFP-PEP injection. No histologic evidence of rejection was observed in these grafts.

### Example 8

### Elimination of Cell Fusion as a Mechanism of Engraftment

To investigate the possibility that cell fusion was the dominant mechanism of hepatic engraftment, the genetic composition of GFP-fluorescing cells in the hepatic parenchyma was analyzed. LCM was used to isolate GFP-expressing cells, and quantitative DNA PCR was used to quantify the genomic DNA contribution from engrafted cells versus wild type cells in the GFP⁺-fluorescing areas of parenchyma. The primers used for the quantitative DNA PCR are presented in Table 3.

TABLE 3

DNA PCR Primer/Fluoroprobe Sets for
Genotyping

| | | |
|---|---|---|
| HrGn | Sequence (5′ to 3′) | SEQ ID NO: |

| | | |
|---|---|---|
| Forward | ATCCTGGTGTACCGCCTGAA | 19 |
| Reverse | TGCTGGATGAAGTGGTACTC | 20 |
| Fluoroprobe | CAGCTGCCACATGCGCACCCT | 21 |
| Hprt | | |
| Forward | GAAGCGAGCCTTTGGTA | 22 |
| Reverse | TGAACTTGAGTTATGGTACCTCA | 23 |
| Fluoroprobe | CTCCGCTCATCTTCCCAGTTCACC | 24 |

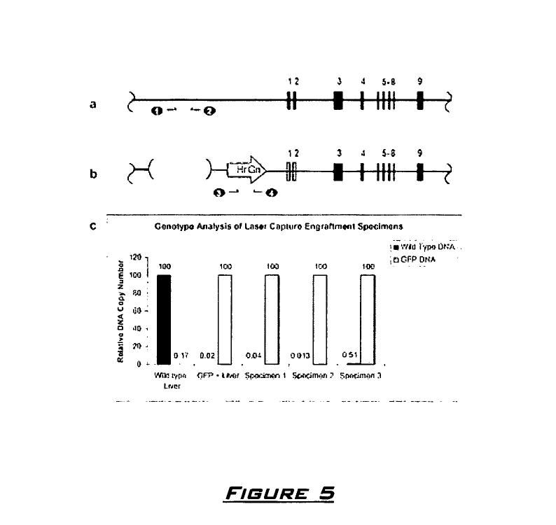

The strategy employed is outlined in FIGS. 5A and 5B, and the results are shown in FIG. 5C. FIGS. 5A-5C depict genetic analysis of laser capture microdissected PEP hepatic engraftment. FIGS. 5A and 5B depict a schematic representation of the wild type Hprt locus (FIG. 5A), and the Hprt locus interrupted with HrGn (GFP) sequences by homologous recombination (FIG. 5B). Wild type hypoxanthine phosphoribosyl transferase (Hprt) sequences are shown by black bars and donor GFP-modified Hprt locus sequences are shown by white bars. FIG. 5C depicts the results of genetic analysis of laser capture microdissected PEP hepatic engraftment from three engraftment specimens (specimens 1, 2, and 3) with wild type liver and GFP liver serving as controls. For the data presented in the bar graph, the wild type sample was set at 100 relative DNA copy numbers for wild type DNA and GFP positive sample was assumed to be 100 relative DNA copy numbers for GFP DNA. Real-time quantitative PCR with primers 1 and 2 can detect only wild type Hprt DNA and PCR with primers 3 and 4 can detect only GFP-modified Hprt locus DNA.

As shown in FIG. 5C, no detectable co-localization of wild type DNA with the GFP DNA was observed in the engraftment areas, thereby excluding cell fusion as the dominant mechanism of the PEP hepatic engraftment and function in our experiments.

### Example 9

### Teratoma Formation in Subjects Injected with PEPs

Teratoma formation is a major concern when using ES-derived cells for cellular engraftment. Including data from 46 C57BL/6 mice and 18 BALB/c mice transplanted with PEPs, an overall teratoma rate of 6.2% (4 of 64 mice) was observed. Three of the 46 injected C57BL/6 mice developed teratoma, and none of them were engrafted mice. Of 18 BALB/c mice injected, an encapsulated teratoma developed in one engrafted mouse, adjacent to the liver but not associated directly with the engrafted regions of the liver.

### Example 10

### Engrafted GFP-PEP Produce F-IX

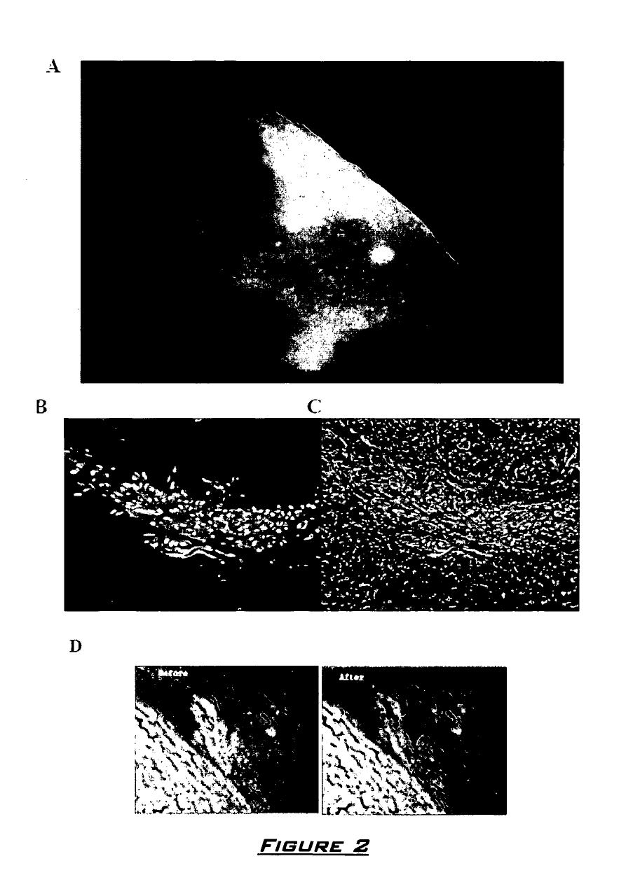

Initial experiments with C57BL/6 F-IX knockout mice, having only minor histocompatibility differences from the strain 129-derived PEPs, showed that of the four mice surviving hepatectomy and injection, two survived beyond 7 days. Mouse No. 1 in FIG. 3 survived for 88 days after injection, achieving a F-IX level of 340 ng/ml, which is 10% of normal. This mouse was 18 months old at the time of death, and postmortem examination revealed no obvious cause of death or pathology related to the PEP engraftment (the lifespan of a F-IX-deficient mouse is about 18-24 months). Mouse No. 2 in FIG. 3 showed typical hepatic histological engraftment with GFP-PEPs and substantial plasma concentrations of F-IX by day 10, but it died during anesthesia for blood drawing on day 10. 18 F-IX-deficient mice were also subjected to partial hepatectomy and injection with human F-IX, but without subsequent PEP injections, none of these mice survived beyond 7 days. Thus, it appears that the injected PEPs provide a survival advantage in this model.

Based on the preceding experiments demonstrating that strain 129-derived GFP-PEPs can engraft across a complete MHC and non-MHC barrier, BALB/c F-IX deficient mice were subjected to PEP transplantation with or without preceding hepatectomy. No immunosuppressive therapy was administered. Four of six mice survived surgery and showed continuous F-IX expression from 38 to 115 days. One mouse (mouse No. 3 in FIG. 3) received hepatectomy before injection, and three mice (mice numbered 4-6 in FIG. 3) received PEP injection without hepatectomy. Mouse No. 3 was sacrificed on day 38 because of the emergence of an abdominal mass adjacent to the liver capsule. Mice Nos. 4-6 were sacrificed on day 115. No evidence of neoplasm was observed in Mice Nos. 4-6 at autopsy or histological sectioning of the explanted livers. GFP mRNA was detectable throughout the livers by PCR.

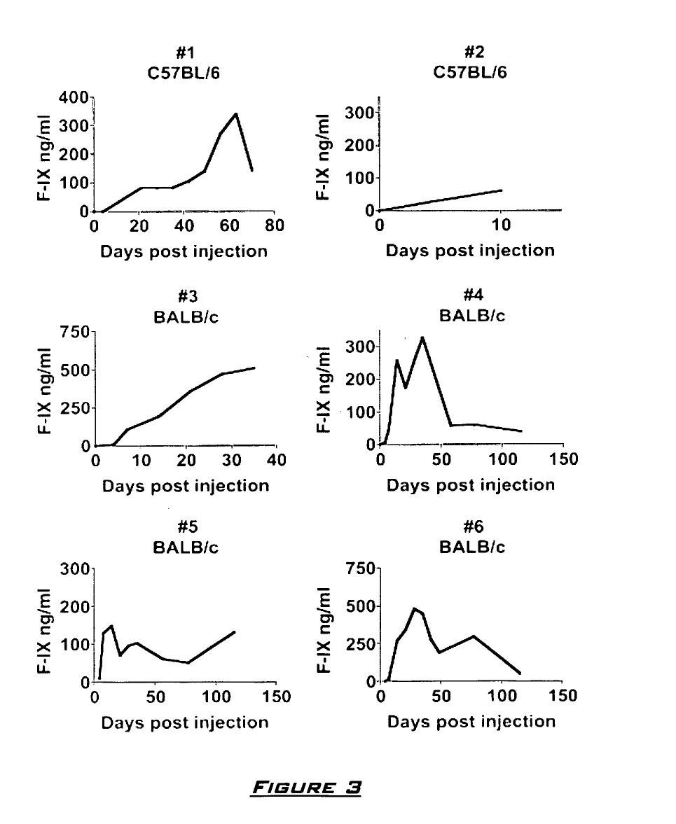

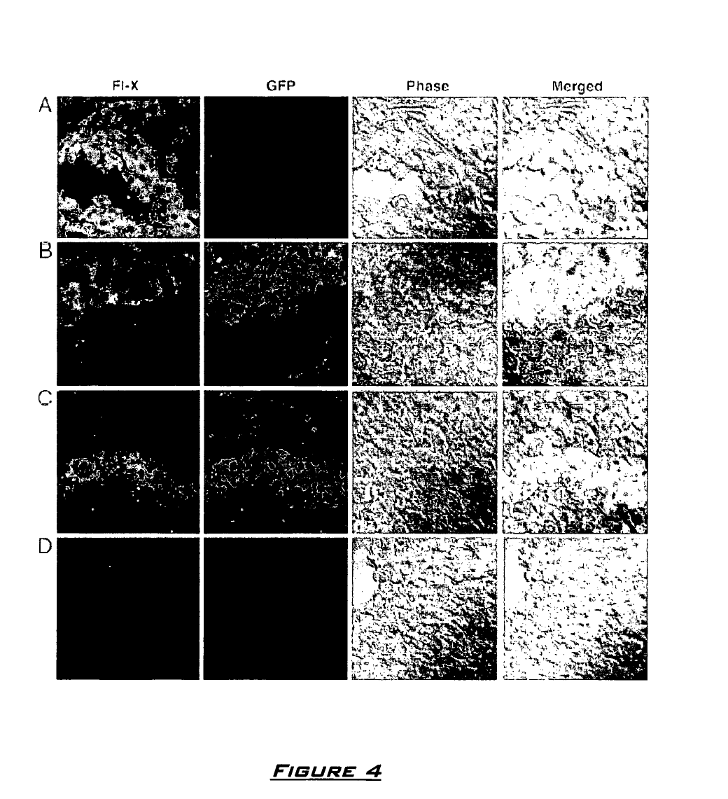

FIG. 4 demonstrates large cords of GFP positive cells within the hepatic parenchyma, from mouse No. 5 in FIG. 3, that co-localize with F-IX by immunofluorescence. Because of this successful engraftment across an MHC barrier, serum from the BALB/c recipients was examined for 129/J-reactive antibodies by flow cytometry at 7 and 115 days after transfer. No detectable antibodies were found at either time that were directed against 129 or BALB/c spleen cells. Normal serum from BALB/c mice was also non-reactive.

### Discussion of Examples 1-10

Directed In Vitro Differentiation of ES Cells into PEPs

It has previously been demonstrated that embryonic chick cardiac mesoderm provides inductive signals in vitro to murine ES cells that results into an immediate differentiation into a putative endodermal phenotype. It was found that cardiac mesoderm induced increased expression of FoxA2 (Hnf-3β), Gata4, and Sox 17α mRNA within two days, and also that this gene expression pattern was consistent with the requirements for endodermal competence found by others in related in situ developmental studies. Thus, it appeared that direct stimulus in vitro of ES cells with fibroblast growth factor might result in similar changes in ES cells in a time frame similar to the development of definitive endoderm in the pre-embryoid body state. When compared to ES cells undergoing spontaneous differentiation after removal from the embryonic fibroblast feeder layer, aFGF (100 ng/ml in media) treated ES cells resulted in a significant reduction in Oct4 mRNA as well as in increase in Afp mRNA. Increased expression of FoxA2, Sox17α, and Gata4 were also seen.

### Cellular Transplantation of PEP

Several investigators have previously described models of ES cell derived engraftment into the liver. Though exciting, these early reports share several common features: (a) low levels of hepatic engraftment; (b) use of ES-differentiated cells obtained from post-embryoid body cultures; and (c) no mechanistic insight into the critical determinants required for engraftment.

In attempting to engraft ES cells early in the endodermal lineage, it was hypothesized that the proliferative capacity of PEPs would be high, and that once engrafted, PEPs would tend to increase the cell mass within the liver. Thus, in order to optimize the potential conditions for engraftment a two-thirds partial hepatectomy model was chosen, which served the dual role of generating temporary hepatic insufficiency as well as a biologic assay for PEP engraftment. Numerous studies have explored possible routes of cell delivery designed for engraftment in the hepatic parenchyma. Cells must traverse the fenestrated sinusoidal endothelium to gain access to the space of Disse where hepatocytes are located.

Dynamic injection studies were performed and revealed that direct injection of PEPs into the liver resulted in the best vascular dispersal throughout the parenchyma where some injected PEPs tended to lodge in the microvasculature. Thus, this method was chosen to provide the most direct delivery of PEPs to the intended engraftment location. Identification of successful engraftment events, from in vivo cell transplant studies, has been hampered by tediously slow experimental throughput, which is dependent on standard histology. To overcome this barrier, BK 4 murine ES cells derived from the A129 mouse were employed that constitutively express a nuclear localizing enhanced green fluorescent protein driven by the β actin promoter (Fair et al., 2003) and three dimensional fluorescence microscopy to screen freshly explanted livers at 10 and 20 days post injection with GFP-PEPs. When fresh explanted liver tissue was screened by 3-D fluorescent microscopy, ES-derived cells were easily visualized and distinguishable from autofluorescence of the hepatic parenchyma, allowing for immediate and accurate detection of engraftment (see FIG. 2A).

By light microscopy, it is very difficult to distinguish the engrafting cells from native hepatocytes, but under fluorescent microscopy, the dominant appearance of GFP-PEP hepatic engraftment displayed a specific and consistent pattern (see FIGS. 2B and 2C). Histologically, GFP-PEP cells engrafted robustly and coursed along the sinusoids within the space of Disse while appearing to be expanding clonally. The appearance of PEP engraftment in liver parenchyma was clearly very different from other reports describing ES derived cells engraftment in the liver.

Observed engraftment in a series of survivors of the hepatectomy and cell injection showed that engraftment at day 3 was characterized by individual or small clusters of engrafted cells. By days 10 and 20, the cell engraftment was typical of that seen in FIGS. 2B and 2C, although the contiguous area of cells was expanded by day 20. Teratoma formation was not considered as engraftment. One animal in the day 10 group and 2 in the day 20 group developed extra-hepatic complex GFP-positive lesions consistent with teratoma. Injection of cells after 7 days of spontaneous differentiation achieved only minimal engraftment in 1 of 4 mice at 10 days. Interestingly, all BALB/c mice showed significant engraftment and no teratomas were seen.

Given robust engraftment and since previous studies had revealed that pluripotent cells engrafted into liver could exhibit allogeneic tolerance, GFP-PEP transplantation across a major MHC barrier was evaluated. Using a similar in vitro and in vivo protocol, BALB/c mice, which are a complete MHC mismatch to the GFP-ES cells from the A129 background, received ⅔ partial hepatectomy followed by cellular transplantation, which resulted in 100% engraftment 10 days following GFP-PEP injection without histologic evidence of rejection.

Cell fusion has been suggested as the dominant mechanism by which stem cells engraft in the liver and numerous elegant studies have shown that stem cells can fuse with endogenous hepatocytes and adopt a liver specific phenotype. These fusion events have been described in engraftment involving individual cells in liver parenchyma rather than the diffuse engraftment of hundreds of cells as in the presently disclosed subject matter. Though fusion as a primary mechanism of such large scale engraftment seemed unlikely, the possibility by analyzing the genetic difference between the GFP⁺ES cells and the wild type host cells was investigated using laser capture microdissection. The resultant HRGn (humanized renilla green) allele copies in extracted engrafted green cells, while not entirely exclusion, were overwhelmingly suggestive that fusion was not the dominant mechanism of engraftment.

### Hepatic Specific Synthetic Function of Engrafted GFP-PEP

Having found stable, robust, and persistent engraftment of GFP-PEPs in hepatic parenchyma, the ability of engrafted GFP-PEPs to function as hepatocytes in vivo was tested. A protocol for performing ⅔ partial hepatectomy and GFP-PEP injection into homozygous Factor IX (F-IX) knockout mice was developed. The Factor IX knockout phenotype in mice, analogous to hemophilia B in humans, is characterized by a severe bleeding diathesis caused the absence of Factor IX protein. This is a very demanding model for ES derived cell hepatic engraftment, as production of biologically active F-IX protein is a complex function that requires synthesis and extensive posttranslational modification of the Factor IX protein, which normally occurs only in the mature hepatocyte lineage. Another advantage is that recombinant human F-IX does not cross react with mouse Factor IX and has a very short half-life in mice. Thus, substantial production of mouse Factor IX would be a robust, real time marker of hepatocyte function derived from engrafted GFP-PEPs.

Factor IX knockout mice used for transplant studies were first confirmed to be F-IX negative by PCR genotyping. In order to perform surgical interventions, mice received 100 iu/kg of recombinant human F-IX i.m. and s.c. after anesthesia, and another subcutaneous dose 4 hours post operatively. When the GFP-PEP injections were performed on post-operative day 3, i.m. and s.c. doses of human F-IX were repeated and ELISA assays were used to determine serum concentrations of murine F-IX. Initial experiments with C57BL/6 F-IX knockout mice showed typical hepatic engraftment with GFP-PEPs, and substantial serum concentrations of F-IX by day 10.

Finally, based on experiments disclosed herein that demonstrated GFP-PEPs engraftment across a major MHC barrier, recipient Factor IX knockout mice on BALB/c background received human F-IX, ⅔ partial hepatectomy, and GFP-PEP cellular transplants in a similar fashion as the F-IX knockout C57BL/6 mice, and no immunosuppressive therapy. These mice also demonstrated substantial mouse F-IX levels by ELISA.

### Strategies for Reducing Teratoma Formation

In order to decrease the incidence of teratoma, 25 animals were observed from 180 to 300 days post injection. Approximately 10 of these showed F-IX activity. When sacrificed, PEP engraftment was observed in the hepatic parenchyma of mice with F-IX activity.

With careful cell culture technique and filtering the cells treated with FGF to a single cell suspension prior to injection, no teratomas were observed after injection during this extended observation period.

### SUMMARY

The present disclosure demonstrates that ES-derived cells with early endodermal characteristics engraft, persist, and function in the liver. The cells referred to herein as PEPs have five features that make them highly promising in this setting. First, PEPs are obtained directly from ES cells by culture with aFGF as an inductive stimulus. Second, when injected into the liver parenchyma, PEPs are able to engraft robustly and restore wild type hepatocellular function in F-IX-deficient mice. F-IX-deficient mice (Greenwood et al., 2003; Lin et al., 1997) have a phenotype that is similar to hemophilia B in humans, characterized by a severe bleeding diathesis caused by absence of F-IX protein (Salier et al., 1990; Snyder et al., 1999). F-IX has a short half-life requiring continuous replacement, and, consequently, any substantial production of mouse F-IX in the deficient mice indicated hepatocyte function derived from the engrafted PEPs. Third, PEPs engrafted and persisted across a complete mismatched MHC barrier. Fourth, PEPs exhibited only a low incidence of teratoma formation. Fifth, PEPs did not require host liver injury or regeneration to engraft and function.

The occurrence of teratoma in recipients is undesirable. It appeared, however, that teratoma formation might be a separate event from that leading to functional PEP engraftment. Thus, in the three cases, it occurred in the mice that had no liver engraftment, and, in the one of 21 mice that had the described sinusoidal engraftment pattern, the teratoma was extrahepatic. If this suggestion is correct, then purification of the differentiated PEPs before injection should reduce this complication. In this regard, it is also important to recall that strain 129 mice, from which the ES cells are derived, are notable for an unusually high incidence of spontaneous teratoma (Stevens, 1973).

Several strategies are available that might decrease or eliminate teratoma formation. One strategy is to use ES cells from another mouse strain that is less prone to spontaneous teratomas. A second strategy is based on the observation of a sharply increased expression of MHC class I antigens on the ES cells during in vitro differentiation. A sorting strategy based on this marker should exclude any undifferentiated ES cells likely to give rise to teratomas. Additionally, it appears that careful cell culture combined with ensuring that the cells administered are in the form of a single cell suspension also reduces the incidence of teratoma formation.

The appearance of PEP engraftment in liver parenchyma as described herein is different from that seen in other reports describing ES-derived cell engraftment in the liver (Chinzei et al., 2002; Yin et al., 2002). While the co-inventors do not wish to be bound by any particular theory of operation, it appears that PEPs and/or their progeny might possess an intrinsic rate of proliferation but that this rate subsides over time, as judged by the histologic appearance of the engraftment at 4 months.

Cell fusion has been suggested as a mechanism by which stem cells engraft in the liver, and numerous studies have shown that stem cells can fuse with the native hepatocytes and adopt a liver-specific phenotype (Willenbring et al., 2004; Wang et al., 2003). These fusion events, however, have been in engraftment that involves individual cells in the liver parenchyma, rather than the diffuse engraftment of hundreds of contiguous cells in islands as disclosed herein. Furthermore, pluripotent fractions of umbilical cord blood can engraft into the liver with low rates of fusion (Newsome et al., 2003; Ishikawa et al., 2003). However, it would appear that fusion as a primary mechanism of the large-scale engraftment described herein is excluded.

### REFERENCES

The references listed below, as well as all references cited in the specification, are incorporated herein by reference to the extent that they supplement, explain, provide a background for, or teach methodology, techniques, and/or compositions employed herein.
Ahmad et al. (1995) 310 Biochem J 427-431.
Alam & Cook (1990) 188 Anal Biochem 245-254.
Ausubel et al. (1989) Current Protocols in Molecular Biology, John Wiley & Sons, New York, N.Y., United States of America.
Ber et al. (2003) 278 J Biol Chem 31950-31957.
Cattaneo et al. (2001) 20 DNA Cell Biol 1-9.
Chambers et al. (2003) 113 Cell 643-55.
Chinzei et al. (2002) 36 Hepatology 22-29.
Cubitt et al. (1995) 20 Trends Biochem Sci 448-455.
Doetschman et al. (1985) 87 J Embryol Exp Morph 27-45.
Evans et al. (1981) 292 Nature 154-159.
Fair et al. (2003) 134 Surgery (St. Louis) 189-196.
Gearing et al. (1989) 7 Biotechnology 1157-1161.
Gerfen & Wheeler (1995) 6 Anim Biotech 1-14.
Gluzman et al. (1988) Viral Vectors, Cold Spring Harbor Laboratory, Cold Spring Harbor, N.Y., United States of America.
Greenwood et al. (2003) 1 J Thromb Haemost 95-102.
Gui et al. (2002) 100 Blood 153-158.
Gupta et al. (1999) 19 Semin Liver Dis 19:15-26.
Hamazaki et al. (2001) 497 FEBS Lett 15-19.
Horb et al. (2003) 13 Curr Biol 105-115.
Ishikawa et al. (2003) 996 Ann NY Acad Sci 174-185.
Jin et al. (2000) 270 Biochem Biophys Res Comm 978-982.
Kim et al, (2002) 99 Proc Natl Acad Sci USA 4602-4607.
Kojima et al. (2003) 9 Nat Med 596-603.
Lin et al. (1997) 90 Blood 3962-3966.
Loo & Cotman (1998) Primary and extended culture of embryonic mouse cells. in Cell Biology: A Laboratory Handbook (J. E. Celis, ed.) Vol. 1, pages 65-72, Academic Press, San Diego, Calif., United States of America.
Martin (1981) 78 Proc Natl Acad Sci USA 7634-7638.
McMahon & Bradley (1990) 62 Cell 1073-1085.
Meivar-Levy et al. (2003) 14 Trends Endocrinol Metabol 460-466.
Miyatsuka et al. (2003) 310 Biochem Biophys Res Comm 1017-1025.
Nagy et al. (1990) 110 Development 815-821.
Nagy et al. (2003) Manipulating the Mouse Embryo: A Laboratory Manual (Third Edition), Cold Spring Harbor Laboratories, Cold Spring Harbor, N.Y., United States of America.
Newsome et al. (2003) 124 Gastroenterology 1891-1900.
Odagiri et al. (1996) 271 J Biol Chem 1909-15.
PCT International Publication No. WO 97/47763
Pease et al. (1990) 141 Dev Biol 344-352.
Pera & Trounson (2004) 131 Development 5515-25.
Qian et al. (1995) 15 Mol Cell Biol 1364-1376.
Robertson (1987) Teratocarcinomas and Embryonic Stem Cells: A Practical Approach IRL Press, Washington, D.C., United States of America.
Rose & Botstein (1983) 101 Methods Enzymol 167-180.
Rosenberg et al. (1990) 323 New Engl J Med 570-578.
Ruhnke et al. (2003) 21 Stem Cells 428-436.
Salier et al. (1990) 265 J Biol Chem 7062-7068.
Sambrook & Russell (2000) Molecular Cloning: A Laboratory Manual (Third Edition), Cold Spring Harbor Laboratory Press, Cold Spring Harbor, N.Y., United States of America.
Sapir et al. (2000) 6 Nat Med 568-572.
Scharfmann et al. (1991) 88 Proc Natl Acad Sci USA 4626-4630.
Simonet et al. (1993) 269 J Biol Chem 8221-8229.
Smith et al. (1988) 336 Nature 688-690.
Snyder et al. (1999) 5 Nat Med 64-70.
Stevens (1973) 50 J Natl Cancer Inst 235-242.
Sylvester & Scholer (1994) 22 Nucleic Acids Res 901-11.
Thomas & Capecchi (1987) 51 Cell 503-512.
Thomson et al. (1998) 282 Science 1145-1147.
Tucker et al. (1997) 25 Nucleic Acids Res 3745-6.
U.S. Pat. No. 6,194,635.
U.S. Pat. No. 6,200,806.
U.S. Pat. No. 6,774,120.
U.S. Pat. No. 6,800,480.
U.S. Pat. No. 6,875,607.
Wang et al. (2003) 422 Nature 897-901.
Willenbring et al. (2004) 10 Nat Med 744-748.
Williams et al. (1988) 336 Nature 684-687.
Williams et al. (1993) 92 J Clin Invest 503-508.
Wilson et al. (1991) Principles of Internal Medicine, McGraw-Hill, New York, N.Y., United States of America.
Yasuda et al. (1998) 273 J Biol Chem 34413-34421.
Yin et al. (2002) 20 Stem Cells (Dayton) 338-346.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method for treating a disease associated with abnormal or missing function of a tissue, the method comprising:
(a) providing a putative endoderm precursor cell (PEP); and
(b) introducing into a subject in need thereof the PEP, whereby the PEP becomes engrafted in an endoderm-derived tissue of the subject and provides a normal function to or replaces the missing function in the subject, or combinations thereof.

2. The method of claim 1, wherein the subject is a mammal.

3. The method of claim 1, wherein the endoderm-derived tissue is liver.

4. The method of claim 1, wherein the PEP becomes stably engrafted into the endoderm-derived tissue.

5. The method of claim 1, wherein the abnormal or missing function of the tissue results from abnormal or absent function of a cell type of the tissue or abnormal or absent expression of a gene of interest in the tissue.

6. The method of claim 5, wherein the gene of interest is normally expressed in liver cells in the absence of the disease.

7. The method of claim 5, wherein the gene of interest is a Factor IX gene.

8. The method of claim 1, wherein providing a PEP comprises differentiating an embryonic stem cell or a pre-embryoid body cell in vitro to form a PEP.

9. The method of claim 8, wherein the differentiating comprises exposing the embryonic stem cell or the pre-embryoid body cell to a growth factor.

10. The method of claim 9, wherein the growth factor is acidic fibroblast growth factor (aFGF).

11. The method of claim 8, wherein the differentiating results in reduced expression of Oct4 and increased expression of an endodermal marker selected from the group consisting of FoxA2 (HNF-β3), Gata4, Sox17α, albumin, and α-fetoprotein in the in vitro differentiated cell.

12. The method of claim 1, wherein the introducing is into liver parenchyma of the subject.

13. The method of claim 1, wherein the in vitro differentiated cell is allogeneic to the subject.

14. The method of claim 1, wherein the in vitro differentiated cell further comprises a coding sequence that is not normally expressed in the liver of the subject in the absence of disease, but is normally expressed in another tissue or cell type of the subject in the absence of disease.

15. The method of claim 1, further comprising performing a partial hepatectomy on the subject prior to the introducing step.

16. A method for producing a reagent comprising an in vitro differentiated cell, the method comprising:
(a) providing an embryonic stem (ES) cell or a pre-embryoid body cell; and
(b) differentiating the ES cell or the pre-embryoid body cell in vitro, wherein the differentiating leads to the ES cell or pre-embryoid body cell:
(i) expressing a marker associated with a differentiated cell of interest;
(ii) differentiating into a cell type of interest; or
(iii) combinations thereof.

17. The method of claim 16, wherein the embryonic stem cell or the pre-embryoid body cell is a mammalian cell.

18. The method of claim 17, wherein the mammalian cell is a human cell.

19. The method of claim 16, wherein the differentiating comprises exposing the embryonic stem cell or the pre-embryoid body cell to a growth factor in an amount and for a time sufficient to provide reduced expression of Oct4 and increased expression of an endodermal marker selected from the group consisting of FoxA2 (HNF-β3), Gata4, Sox17α, albumin, and α-fetoprotein in the embryonic stem cell.

20. The method of claim 19, wherein the growth factor is an acidic fibroblast growth factor (aFGF).

21. A method for generating a putative endoderm precursor cell (PEP) from an embryonic stem cell, the method comprising:
(a) culturing the embryonic stem cell in the absence of feeder cells and LIF; and
(b) exposing the embryonic stem cell to acidic fibroblast growth factor in an amount and for a time sufficient to provide reduced expression of Oct4 and increased expression of an mRNA selected from the group consisting of α-fetoprotein, Gata4, albumin, FoxA2 (HNF-β3), and Sox 17α in the embryonic stem cell, whereby a PEP is generated.

22. The method of claim 21, wherein the embryonic stem cell is a mammalian embryonic stem cell.

23. The method of claim 22, wherein the embryonic stem cell is a 1 human embryonic stem cell.

24. The method of claim 21, wherein the embryonic stem cells are exposed to acidic fibroblast growth factor in an amount of about 100 ng/ml in a culture medium for about 7 days.

25. The method of claim 21, further comprising purifying the PEP.

26. A putative endoderm precursor cell (PEP), wherein:
(a) the PEP is an in vitro differentiated derivative of an embryonic stem cell; and
(b) expresses a reduced level of Oct4 and an increased level of an mRNA selected from the group consisting of α-fetoprotein, Gata4, albumin, FoxA2 (HNF-β3), and Sox 17α as compared to the embryonic stem cell from which is was derived.

26. The cell of claim 25, where the cell is capable of engraftment of an endoderm-derived tissue of a subject when administered into the endoderm-derived tissue.

27. The cell of claim 26, wherein the engraftment comprises long term engraftment.
